(19) United States
(12) Patent Application Publication
(10) Pub. No.: US 2003/0212123 A1
(43) Pub. Date: Nov. 13, 2003

(54) COX-2 selective carprofen for treating pain and inflammation in dogs

(75) Inventors: Kristin Lundy DeMello, Ledyard, CT (US); Anthony P. Ricketts, Stonington, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/422,220

(22) Filed: Apr. 24, 2003

Related U.S. Application Data
(62) Division of application No. 09/308,955, filed on May 27, 1999.
Application No. 09/308,955 is a 371 of international application No. PCT/IB98/00662, filed on May 1, 1998.
(60) Provisional application No. 60/045,635, filed on May 5, 1997.

(51) Int. Cl. A61K 31/403

(52) U.S. Cl. 514/411

(57) ABSTRACT

Treating or preventing inflammatory processes and diseases in dogs associated with the activity of inducible cyclo-oxygenase-2 (COX-2), while at the same time reducing or eliminating undesirable side effects associated with simultaneous inhibition of the activity of constitutive cyclo-oxygenase-1 (COX-1) by selectively inhibiting COX-2 activity with reference to COX-1 activity, wherein the selectivity ratio or COX-2:COX-1 activity inhibition is at least 3:1 based on ex vivo inhibition levels measured in whole blood; the inhibitor is a member selected from the group of anti-inflammatory compounds consisting essentially of salicylic acid derivatives, p-aminophenol derivatives, indole and indene acetic acids, heteroaryl acetic acids, arylpropionic acids, anthranilic acids, enolic acids, and alkanones; the inhibitor in particular is comprised of (+)(S)-enantiomer of 6-chloro-α-methyl-9H-carbazole-2-acetic acid.

### FIELD OF THE INVENTION

The present invention concerns the treatment of pain and inflammation in dogs with anti-inflammatory agents which are non-steroidal anti-inflammatory drugs (NSAIDs), and in particular such agents having a reduced incidence of adverse gastrointestinal side effects, since such side effects are a prevalent and potentially severe problem in dogs.

### BACKGROUND OF THE INVENTION

As is well known to artisans of ordinary skill in this field, e.g., veterinarians, the canine species, ie., dogs, especially older dogs, are very susceptible to chronic inflammatory processes such as degenerative joint disease. Because of the very large number of dogs which are kept as pets or for utilitarian purposes such as guard dogs and seeing-eye dogs, there has been an ongoing effort to find pharmaceutical agents which will impede or altogether stop the progress of such inflammatory disease processes in dogs, or at least ameliorate the symptoms of the inflammation such as pain and edema. One class of such pharmaceutical agents which has been investigated extensively for anti-inflammatory and analgesic use in humans, and more recently in dogs, is that of the non-steroidal anti-inflammatory drugs (NSAIDs). This type of anti-inflammatory drug has been explored widely and new, improved agents of this type for use in humans have been discovered and developed over a period of decades.

However, the use of NSAIDs in dogs has been more limited, e.g., only two such NSAIDs have been approved by the Food and Drug Administration, Committee on Veterinary Medicine (FDA/CVM), for use in dogs in the United States, i.e., ARQUEL®, meclofenamic acid, and RIMADYLOR, carprofen. Consequently, there is less experience and knowledge in veterinary medicine about safety and efficacy issues surrounding the use of NSAIDs in dogs. In veterinary medicine, for example, the most common indication for NSAIDs is the treatment of degenerative joint disease (DJD), which in dogs often results from a variety of developmental diseases, e.g., hip dysplasia and osteochondrosis, as well as from traumatic injuries to joints. In addition to the treatment of chronic pain and inflammation, NSAIDs are also useful in dogs for treating post-surgical acute pain, as well as for treating clinical signs associated with osteoarthritis.

This demand for canine NSAID therapy, combined with the absence of any approved NSAIDs for this purpose, has resulted in substantial off-label use in dogs of NSAIDs approved for humans, sometimes with disastrous consequences. The veterinary literature is replete with reports of gastrointestinal hemorrhage, perforation and peritonitis in dogs associated with the use of NSAIDs approved for human use such as indomethacin, naproxen, aspirin, ibuprofen, and phenylbutazone. Although such gastrointestinal adverse reactions afflict human patients as well, dogs often receive inappropriately high doses because of the lack of information about proper dosing, and because of the inherently high degree of canine susceptibility to such gastrointestinal adverse reactions. There is, accordingly, a pressing need for safe yet effective NSAIDs in the treatment of pain and inflammation in dogs.

While the search for safe and effective NSAID agents in canine therapy must deal with the potential for serious adverse gastrointestinal reactions, other adverse reactions include kidney and liver toxicity. However, the most serious of these are the gastrointestinal effects such as single or multiple ulcerations, including perforation and hemorrhage of the esophagus, stomach, duodenum or small and large intestine. These adverse reactions are usually debilitating, but can often be severe, and occasionally can even be life-threatening. Indeed, the therapeutic index for the use of NSAIDs in dogs can be so low as to contraindicate such treatment.

The expression “therapeutic index” is sometimes generally defined as the ratio of the LD₅₀ to the ED₅₀ of a drug, and is intended to be a statement of how selective the drug is in producing its desired effects. As used herein, however, the expression “therapeutic index” is more consistent with the definition utilized in the animal health field, which is the ratio of the maximum tolerated dose in the animal to the minimum effective dose in the animal. In the present invention the term “animal” refers, of course, to dogs. The maximum tolerated dose in a particular canine subject would typically be determined by a number of different assays and techniques. For example, gastrointestinal hemorrhage may be determined by assay methods commonly used to detect occult blood in stool specimens, while endoscopy can be used to detect the occurrence of ulceration or perforation. Where the animal is euthanized as part of the study, autopsy can provide valuable information as well.

It has been the expectation in the art heretofore that any NSAID candidate, especially one for canine therapy, would have a low therapeutic index. The hope has always been that the therapeutic index was not so low so as to render the candidate unacceptable for use in dogs. Thus, an important aspect of the present invention was the surprising discovery that the anti-inflammatory compositions described herein have an extremely high therapeutic index when used for treating pain and inflammation in dogs, and further that said anti-inflammatory compositions have unique possession of this unexpected property, virtually to the exclusion of all other NSAIDs.

A significant body of knowledge has accumulated in recent years regarding the mechanisms of action whereby NSAIDs achieve their anti-inflammatory therapeutic results, as well as whereby they produce serious gastrointestinal adverse reactions at the same time. While most of this body of knowledge has been gathered with respect to NSAID mechanisms of action in humans, it is applicable to a large extent to the same mechanisms of action in dogs, although there is apparently some species specificity, as is further below-detailed. With regard to the therapeutic efficacy of NSAIDs, it has long been known that the mechanism of action whereby NSAIDs reduce inflammation is their ability to disrupt the arachidonic acid cascade, which leads to the endogenous production of prostaglandins, thromboxanes and leukotrienes. These lipid compounds are referred to collectively as “eicosanoids” because they are commonly derived from C₂₀ polyunsaturated fatty acids, the eicosenoic acids, the most abundant of which is arachidonic acid. Arachidonic acid, which is cis-Δ⁵, cis-Δ⁸, cis-Δ¹¹, cis-Δ¹⁴ eicosatetraenoic acid, is the dominant precursor for many prostaglandins and leukotrienes which are mediators of inflammation.

In the first stage of the arachidonic acid cascade, arachidonic acid is released as a result of tissue-specific stimuli by hormones or proteases, or by membrane perturbance, and involves the action of a specific phospholipase A₂. A free arachidonate results which in the second stage of the cascade is acted on by the bifunctional enzyme prostaglandin endoperoxide synthase, also referred to as prostaglandin H synthase (hereafter PGH synthase), the first activity of which is as a cyclo-oxygenase, while the second activity involves a two-electron reduction. Most NSAIDs act as inhibitors of the cyclo-oxygenase activity of PGH synthase, and thereby block the production of various prostaglandins, which are locally acting hormones which carry out their functions by binding to specific cellular receptors. The prostaglandins are very potent but are also quickly catabolized. Some of these prostaglandins are mediators of the inflammatory process; however, some of these prostaglandins also have a gastrointestinal protective function. Blocking production of these beneficial prostaglandins is one of the chief factors contributing to the adverse gastrointestinal reactions which are experienced with the use of NSAID therapy. Accordingly, there has been an ongoing search for pharmaceutical agents which, while acting as cyclo-oxygenase inhibitors, also by some additional mode of action or another, have substantially reduced gastrointestinal adverse reactions and resulting side effects.

It has more recently been discovered that in humans and virtually all other mammalian species which have been studied, that cyclo-oxygenase (COX) comprises two isozymes, a constitutive enzyme (COX-1) and an inducible enzyme (COX-2), which have different activities in various systems. The identification of the COX-2 isozyme led to conjecture early on that it might be responsible for the production of prostaglandins exclusively or primarily at inflammatory sites. Since this has now been shown to be the case, the selective inhibition of the COX-2 isozyme will reduce inflammation without the side effects of gastrointestinal toxicity. COX-1 and COX-2 have a 60% homology, similar Kₘ values, and the same arachidonic acid binding sites, but COX-2 accepts a wider range of substrates than does COX-1.

Another metabolic pathway leads from the above-mentioned arachidonate to the production of leukotrienes through the action of a lipoxygenase. Some of these leukotrienes are also mediators of inflammation; accordingly, much effort has been expended in the search for pharmaceutical agents which are dual inhibitors of both cyclo-oxygenase and lipoxygenase.

A particular NSAID which has been used to treat inflammatory diseases in dogs, and the only one of two up to the present time which has been approved for use in the United States for that purpose by the Food and Drug Administration, Committee on Veterinary Medicine (hereafter FDA/CVM), is the above-mentioned carprofen. Carprofen, racemic 6-chloro-α-methylcarbazole-2-acetic acid, belongs to the aryl propionic acid class of NSAIDs. Other members of this class are, e.g., benoxaprofen, cicloprofen, fenoprofen, flurbiprofen, furaprofen, indoprofen, ketoprofen, pirprofen, and suprofen. While these compounds are closely related in structure, they may still possess different anti-inflammatory and other biological properties. Carprofen, for example, has been shown to be a relatively weak inhibitor of cyclo-oxygenase, but has also been shown, in humans and various animal models, to decrease significantly the pain and swelling, and other symptoms of inflammation. These evaluations of carprofen are described in the technical literature, some of which is cited and discussed further below. Carprofen has also been shown to be inactive with respect to lipoxygenase in the rat, and does not, presumably, block production of leukotrienes. While the mode of action of carprofen still appears to be unknown, it has been demonstrated to have some activity against phopholipase A₂.

### DESCRIPTION OF THE PRIOR ART

The current state of knowledge in the art, as shown by the disclosures of the below-discussed references, has been largely confused when trying to explain the mechanisms of action in dogs whereby carprofen is able to possess good anti-inflammatory activity while at the same time exhibiting diminished adverse gastrointestinal and other reactions. The prior art has characterized carprofen as having weak to no cyclo-oxygenase inhibitor activity, and has concluded that it must, therefore, be operating by some altogether different mechanism of action.

As already mentioned further above, recently the existence of the constitutive COX-1 and inducible COX-2 isozymes has been reported, including their diverse roles in protecting the gastrointestinal mucosa and in mediating inflammation, respectively. This has led, naturally, to the investigation of compounds in a search for any which might, when used in dogs as well as other animals and humans, inhibit only the inducible COX-2 isozyme, i.e., be selective COX-2 inhibitors. These investigations have included, in particular, evaluation of the inhibitory activity of the enantiomers of various NSAIDs, including especially ketoprofen, ketorolac, and flurbiprofen. These particular NSAIDs were examined for differences in inhibitory potency in terms of one enantiomer vs. the other, as well as inhibitory potency in the case of each enantiomer treated separately, against the COX-1 enzyme as compared to inhbitory potency against the COX-2 enzyme. The results of these investigations showed that for all three NSAIDs, the enantiomers were equally potent against both the COX-1 and COX-2 enzymes. Thus, neither the R- nor the S-enantiomer of any of these NSAIDs showed any selectivity toward COX-1 or COX-2. While indeed there were differences in potency between the enantiomers, with the S-enantiomer being the more potent in each case, with regard to COX-1 vs. COX-2 inhibition, each enantiomer showed equal inhibitory potency, i.e., neither enantiomer was able to discriminate between the two isozymes.

Accordingly, as a result of these investigations in the prior art, the current state of the art is that neither carprofen, nor any of the other classical NSAIDs having a carboxylic acid moiety, have been found to be selective COX-2 inhibitors in dogs or any other species. These conclusions have been reinforced by the disclosure in the prior art of the conformation of the sequenced structures of the COX-1 isozyme, as well as of the COX-2 isozyme, complexed with various inhibitors, at the level of their basic functional molecular configurations. As a result of these reported studies, the art now teaches that carboxylic-acid-group-containing inhibitors such as carprofen are inherently incapable of being selective COX-2 inhibitors. Thus far, only sulfonyl-moiety-containing compounds and nabumetone, a naphthalenyl-2-butanone compound, have been reported to be selective inhibitors of the COX-2 isozyme.

Also recently in the art, with the above-mentioned discovery of the existence of the constitutive COX-1 and inducible COX-2 isozymes, studies have been conducted using various species in order to ascertain the existence of any stereoselective inhibition specific to one or more of said species. These investigations of the individual activity of the R- and S-enantiomers of certain NSAIDs on COX-1 vs. COX-2 inhibition in various species have reported that there is a consistent potency difference between R- and S-enantiomers of all chiral NSAIDs investigated. However, these studies have also reported that there is no species specific selectivity by either the R- or the S-enantiomer of any of the chiral NSAIDs investigated, with respect to COX-1 vs. COX-2. For example, it has been shown that while the S-enantiomer is, e.g., three-times as potent as the R-enantiomer in inhibiting both the COX-1 and the COX-2 isozymes in a given species, that with respect to either of these same isozymes, the S-enantiomer shows equal potency in inhibiting both the COX-1 and the COX-2 isozymes, i.e., the S-enantiomer shows no selective inhibition of COX-2 in that species.

In view of the above-described state of the art, especially where dogs have been the species investigated, it was wholly unexpected that, in accordance with the present invention, carprofen has been found to be a surprisingly potent inhibitor of the COX-2 isozyme in the dog; and further that it is a selective COX-2 inhibitor in the dog. Moreover, the selectivity of carprofen against COX-2 is as much as two fold greater than that of virtually all other NSAIDs, including carboxylic-acid-moiety-containing NSAIDs, and purportedly COX-2 selective sulfonyl-moiety-containing NSAIDs. The selection of carprofen as the preeminent selective inhibitor of the COX-2 isozyme in dogs, from among all other NSAIDs, not only runs counter to the current teachings in the art, but is also a wholly unexpected discovery in terms of the surprising results achieved. The fact that all of the representative other NSAIDs evaluated herein have been approved for administration to humans in the United States, including even those that are presently available in commerce, lends further credence to the soundness of these conclusions. Carprofen stands out from all of the other NSAIDs evaluated herein, including those which have been approved for human use or evaluation in clinical trials.

In view of the above-described state of the art, especially where dogs were the species investigated, it was further wholly unexpected that, in accordance with the present invention, the S-enantiomer of carprofen has been found to be a highly selective inhibitor of the COX-2 isozyme vs. the COX-1 isozyme in the dog, and that this is the case to a significantly greater extent than all other NSAIDs or their S-enantiomers, including carboxylic-acid-moiety-containing NSAIDs, and purportedly COX-2 selective sulfonyl-moiety-containing NSAIDs. The selection of the S-enantiomer of carprofen as the preeminent and selective inhibitor of the COX-2 isozyme in dogs, which at the same time exhibits little or no adverse gastrointestinal or other reactions in dogs, from among all other NSAIDs, not only runs counter to the current teachings in the art, but is also a wholly unexpected discovery in terms of the surprising results achieved. The fact that all of the representative other NSAIDs evaluated herein have been approved for administration to humans in the United States, including even those that are presently available in commerce lends further credence to the soundness of these conclusions.

In view of the above-described state of the art, especially where dogs were the species investigated, it was still further wholly unexpected that, in accordance with the present invention, the S-enantiomer of carprofen has been found to have, by reason of its being a highly selective inhibitor of the COX-2 isozyme, a surprisingly improved level of anti-inflammatory, analgesic and anti-pyretic activity compared to that of all other NSAIDs, including those having a carboxylic-acid-moiety, or their S-enantiomers, as well as a surprisingly reduced level of adverse gastrointestinal and other reactions compared to that of all other NSAIDs, including those having a carboxylic-acid-moiety, or their S-enantiomers.

As already mentioned, carprofen belongs to the aryl propionic acid class of NSAIDs, and is as well a member of the subclass of such compounds which are substituted carbazole acetic acids. These compounds and their use as anti-inflammatory, analgesic and anti-rheumatic agents are described in U.S. Pat. No. 3,896,145. The anti-inflammatory activity of carprofen in dogs was investigated by McKellar et al. and reported in “Pharmacokinetics, tolerance and serum thromboxane inhibition of carprofen in the dog”, Journal of Small Animal Practice, 31, 443-448, 1990. The biological activities of the individual (−)(R) and (+)(S) enantiomers of carprofen, as well as of racemic carprofen in dogs were further investigated by McKellar et al. and reported in “Stereospecific pharmacodynamics and pharmacokinetics of carprofen in the dog”, J. Vet. Pharmacol. Therap. 17, 447-454, 1994. Reporting on the results of this investigation, the authors concluded that:

“[t]he mode of action of CPF [carprofen] remains unknown . . . . CPF administered as a racemic mixture or as either the (+)(S) or (−)(R) enantiomer did not significantly inhibit the generation of T×B₂ from blood or PGE₂ and 12-HETE in inflammatory exudate, suggesting that it does not act as a conventional NSAID.

A principal mode of action of most NSAIDs is known to be inhibition of the enzyme cyclooxygenase in the generation of inflammatory prostaglandins from arachidonic acid . . . . The large number of mediators now known to be involved in inflammation provide many possible targets for anti-inflammatory drugs, and it is probable that CPF has its principal action activity on one or a number of mediators not yet identified.”

Thus, it was concluded by these workers in the art that carprofen has a mode of action which is “not primarily attributable” to cyclo-oxygenase inhibition; and indeed, that the “poor activity of carprofen against cyclo-oxygenase, lipoxygenase and phospholipase suggest that its major mode of action may be by mechanisms other than eicosanoid inhibition.” Such findings and conclusions have instructed other workers in the art away from the surprising discovery of the present invention, that carprofen dosed dogs show near total inhibition of PGE2 synthesis, as is demonstrated further below in a working example.

Another study of carprofen in dogs was reported by Holtsinger et al. in “The Therapeutic Efficacy of Carprofen (Rimadyl-V™) in 209 Clinical Cases of Canine Degenerative Joint Disease”, V.C.O.T. 1992; 5: 140-4. The authors theorized from in vitro studies that carprofen might exert its anti-inflammatory action, at least in part, by inhibiting neutrophil migration, and that this might explain how carprofen could be equipotent with indomethacin as an anti-inflammatory agent, and yet have an ulcerogenic potential which was 16 times less. However, the authors voiced many reservations regarding their conclusions and thereby failed to point in the direction of the discovery on which the present invention is based.

A study of the use of carprofen to treat osteoarthritis in dogs was reported by Vasseur et al. in “Randomized, controlled trial of the efficacy of carprofen, a nonsteroidal anti-inflammatory drug, in the treatment of osteoarthritis in dogs”, J Am Vet Med Assoc, 206(6): 807-811, 1995. These investigators were also unable to explain the activity of carprofen. They concluded, on the one hand, that prostaglandins are protective of the gastrointestinal mucosa, and that “carprofen, like other NSAID, inhibits prostaglandin synthetase, blocking prostaglandin biosynthesis.” However, this conclusion was inconsistent with the results of their study, which determined that “carprofen has minimal or no harmful effects on the gastrointestinal mucosa in dogs.”

A study of the use of carprofen to treat acute postoperative pain in dogs was reported by Lascelles et al. in “Postoperative analgesic and sedative effects of carprofen and pethidine in dogs”, Veterinary Record, 134: 187-191, 1994. These investigators were similarly confused as to the mode of action of carprofen, noting on the one hand that “Carprofen . . . at therapeutic doses, seems to be a poor inhibitor of prostaglandin synthetase (or cyclo-oxygenase), the enzyme responsible for the synthesis of inflammatory mediators produced by tissue damage”, while acknowledging on the other hand that “[n]evertheless, studies have shown it to be a good analgesic for both acute and chronic pain.”

A much earlier study which compared the biological activities of indomethacin to those of the stereoisomers and racemate of carprofen in humans was done by Gaut et al. and reported in “Stereoisomeric Relationships Among Anti-Inflammatory Activity, Inhibition of Platelet Aggregation, and Inhibition of Prostaglandin Synthetase”, PROSTAGLANDINS, Vol. 10, NO. 1, July 1975. The study concluded that the carprofen racemate, unlike indomethacin, would have no effect on platelet aggregation and thus would produce no prolongation of bleeding time at doses possessing anti-inflammatory activity. The data from the study also suggested that the carprofen racemate and [S] isomer have greater specificity toward anti-arthritic activity and are less ulcerogenic than indomethacin.

A portion of the advances in prostaglandin research which were presented in 1994 at the 9th International Conference on Prostaglandins and Related Compounds focused on COX-2 selectivity. A meeting report presented by Battistini et al. entitled “COX-1 and COX-2: Toward the Development of More Selective NSAIDs” and published in DN&P, 7(8), October 1994, contained comparative data obtained from a large variety of cell types, including human, mouse or rat types, using different stimuli, as reported in the technical literature reviewed and cited by Battistini et al. Data demonstrating IC₅₀ values against COX-1 and COX-2 for numerous anti-inflammatory compounds, including carprofen, was presented, and the ratio of COX-2/COX-1 was used to determine COX-2 selectivity. Inverting the ratios reported by Battistini et al. so as to be consistent with those used herein to facilitate comparison, the most COX-2 selective compounds had ratios of 1428.57 to 50,000.00. The IC₅₀ (μM) values of carprofen for both the COX-1 and COX-2 isozymes were shown as being exactly the same (10.96) to give a ratio of 1.00, clearly demonstrating that carprofen has no COX-2 selectivity. The values cited by Battistini et al. were originally reported by Akarasereenont et al. in “Relative Potency of Nonsteroid Anti-inflammatory Drugs As Inhibitors of Cyclo-oxygenase-1 or Cyclo-oxygenase-2”, Br. J. Pharmacol., Proceedings Supp. No. 183P, Jan. 5-7, 1994; and by the same group in Mitchell et al., “Selectivity of nonsteroidal antiinflammatory drugs as inhibitors of constitutive and inducible cyclooxygenase”, Proc Nat Acad Sci USA, 90: 11693-7, 1993. The IC₅₀ (μg/mL) values (n=9) of carprofen inhibition of COX-1 (derived from bovine aortic endothelial cells) and COX-2 (derived from lipopolysaccharide stimulated J774.2 macrophages) were 3±0.41 and 3±1.72, respectively, to give a ratio of 1.00. This is in complete contrast to the data for several compounds which were shown to have from 1000 to 4000 times more selectivity for COX-2. The report observed that none of the selective COX-2 inhibitors described are carboxylic acids, like the vast majority of existing NSAIDs, including carprofen. Indeed, all of the COX-2 selective inhibitors have a sulfonyl group in the molecule, which in the case of meloxicam is incorporated into its 1,2-benzothiazine-1,1-dioxide ring structure. If this correlation holds, the report speculates, the Arg 150 residue of the COX protein would not be an essential binding site for selective inhibitors of COX-2. Arg is essential for COX activity because it binds the terminal carboxyl group of arachidonic acid, and is thus most likely the binding site for the carboxylic acid functional group of most existing NSAIDs. Accordingly, an inhibitor which was selective for COX-2 would be expected to bind to a feature which was unique to the COX-2 isozyme protein structure, and not to a feature which was common to both the COX-1 and COX-2 isozyme protein structures.

An even more specific interpretation of the molecular interactions of the classical NSAIDs and COX-2 selective inhibitors with the protein structure of the PGH synthase enzyme, more commonly known as the cyclo-oxygenase enzyme, was reported by Kurumbail et al. in “Structural Basis for Selective Inhibition of Cyclo-oxygenase-2 by Anti-Inflammatory Agents”, NATURE 384, 644-648, December 1996. This interpretation is based on the reported structures of unliganded murine COX-2 and complexes with flurbiprofen, indomethacin, and SC-558, a selective COX-2 inhibitor having a phenylsulfonamide group but no carboxylic acid group, determined at 3.0 to 2.5 Å resolution.

The subject report indicates that the human and murine COX-2 enzymes are expected to be very similar because of the 87% identity and strict sequence conservation in the cyclo-oxygenase active site. Flurbiprofen, a slow-binding competitive inhibitor of both COX-1 and COX-2, binds in the long hydrophobic channel and excludes substrate from the cyclo-oxygenase active site. SC-558 is a diaryl heterocyclic inhibitor with a central pyrazole ring and a sulfonamide group attached to one of the aryl rings. In COX-2 the channel that leads from membrane to the cyclo-oxygenase active site forks at the SC-558 binding site, with one branch forming a cavity that accepts the bromophenyl ring of SC-558, while the other branch forms a pocket which is virtually inaccessible in the COX-1 structure, but which accommodates the entire phenylsulfonamide moiety in COX-2. This pocket is more accessible in COX-2 because valine is substituted for isoleucine, which has a larger side chain, at position 523. Access of the phenylsulfonamide group to the new pocket in COX-2 is facilitated by another isoleucine to valine change at position 434, which forms a molecular gate extending across the new hydrophilic pocket. Finally, at position 513 histidine in COX-1 is replaced by arginine in COX-2, and superposition of the two enzymes suggests that the imidazole ring of histidine in the COX-1 enzyme would not extend sufficiently for direct interactions with the sulfonamide group of SC-558, as is the case with arginine in the COX-2 enzyme. The subject report notes that in each of the above-described three instances, the inhibition profile of COX-2 is altered dramatically by mutation of a single amino acid.

The subject report goes on to conclude that it appears probable that the primary determinant of COX-2 selectivity in the diaryl heterocyclic class of inhibitors to which SC-558 belongs is the phenylsulfonamide moiety. However, the absence of a carboxylate group is also significant. The arginine at position 120 with its guanidinium group is one of the few charged residues in the hydrophobic cyclo-oxygenase channel, and it stabilizes the carboxylate of classical NSAIDs such as flurbiprofen by way of charge-charge interaction. The absence of such a carboxylate group in SC-558 is probably also a significant component of its COX-2 selectivity. This conclusion is supported by the results of attempts to improve its potency against COX-2 by incorporating an acidic group on the pyrazole of the diaryl heterocyclic structure, which has consistently led to poor selectivity.

The subject report embodies the first example of a membrane protein being successfully studied as a target in structure-based drug design, and approximates the current state of the art concerning the structure/activity relationships of NSAIDs to the cyclo-oxygenase isozymes and their resulting anti-inflammatory activity vs. their adverse gastrointestinal reactions. It is within the context of this state of the art that the present invention will be seen to be a wholly unexpected development which provides, surprisingly, an optimum efficacy/safety profile for the treatment of inflammation in dogs.

The possibility of enantioselective inhibition of the COX-2 isozyme was investigated and reported by Carabaza et al. in “Stereoselective Inhibition of Inducible Cyclooxygenase by Chiral Nonsteroidal Antiinflammatory Drugs”, J Clin Pharmacol 1996; 36:505-512. The stereoselective inhibition of COX-2 by ketoprofen, flurbiprofen, and ketorolac was studied in three different in vitro systems, and the results were compared with the inhibition of COX-1 in three parallel in vitro models. It was found that both isozymes were inhibited by the S-enantiomers of all three NSAIDs on an equal potency basis; but that the R-enantiomers of all three NSAIDs inhibited both isozymes with significantly and correspondingly less potency. Put another way, all three R-enantiomers exhibited equal potency in inhibiting both COX-1 and COX-2, but all of their potency levels were much lower than those of the corresponding S-enantiomers in all cases. The “significant degree of enantioselectivity” referred to in this reported study refers only to R- vs. S-, and does not refer to the COX-2 selectivity uniquely exhibited by the S-enantiomer of carprofen, in accordance with the present invention.

### SUMMARY OF THE INVENTION

There is provided in accordance with the present invention a method of treating or preventing pain and inflammatory processes and diseases associated with the activity of inducible cyclo-oxygenase-2 (COX-2) in a member of the species Canis familiaris in need of such treatment, while at the same time reducing or eliminating undesirable side effects associated with simultaneous inhibition of the activity of constitutive cyclo-oxygenase-1 (COX-1) by selectively inhibiting COX-2 activity with reference to COX-1 activity, wherein the selectivity ratio of COX-2: COX-1 activity inhibition is at least 3:1 based on ex vivo inhibition levels in whole blood measured at a dose giving ≧80% COX-2 inhibition, and preferably ≧90% COX-2 inhibition, comprising administering to said member of the species Canis familiaris an amount therapeutically effective for treating pain and inflammation in accordance with the above-recited limitations, of an anti-inflammatory selective COX-2 inhibitory compound comprising a member selected independently from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones.

It is also within the scope of the present invention to carry out the above-described method of treating or preventing pain and inflammatory diseases and processes by administering more than one member selected from the above-recited group of anti-inflammatory compounds. The desirability of any one such combination of anti-inflammatory active agents will probably be based on obtaining a favorable balance of the pharmacokinetic properties of the individual agents involved. For example, rapidity of onset of action may be balanced with extended therapeutic half-life, or a tendency toward the formation of large cellular reservoirs in certain tissues may be balanced with higher rates of plasma protein binding. All such combinations are contemplated to be within the scope of the present invention.

The present invention provides any anti-inflammatory selective COX-2 inhibitor satisfying the above-described limitations. Among such selective inhibitory compounds is a preferred subgenus of carprofen compounds useful in treating or preventing pain and inflammatory processes and diseases in a member of the species Canis familiaris of the formula:

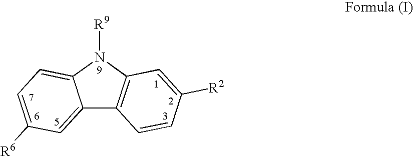

wherein:

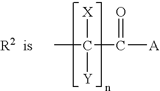

where A is hydroxy, (C₁-C₄)alkoxy, amino, hydroxy-amino, mono-(C₁-C₂)alkylamino, di-(C₁-C₂)alkylamino; X and Y are independently H or (C₁-C₂)alkyl; and n is 1 or 2;

R⁶ is halogen, (C₁-C₃)alkyl, trifluoromethyl, or nitro;

R⁹ is H; (C₁-C₂)alkyl; phenyl or phenyl-(C₁-C₂)alkyl, where phenyl is optionally mono-substituted by fluoro or chloro; —C(═O)—R, where R is (C₁-C₂)alkyl or phenyl, optionally mono-substituted by fluoro or chloro; or —C(═O)—O—R¹, where R¹ is (C₁-C₂)alkyl;

where X and Y are different, the (−)(R) and (+)(S) enantiomers thereof; and all pharmaceutically acceptable salt forms, prodrugs and metabolites thereof which are therapeutically active for treating or preventing pain and inflammation. Where the inhibitor of Formula (I) exists as (−)(R) and (+)(S) enantiomers, in accordance with the present invention there is provided the (+)(S) enantiomer alone, or where both enantiomers are present together, there is provided a racemic or a non-racemic mixture thereof.

The present invention further provides in a preferred aspect of the above-described method of treating or preventing pain and inflammatory processes and diseases in a member of the species Canis familiaris in need of such treatment, administration to said member of an amount therapeutically effective for treating pain and inflammation, of an anti-inflammatory selective COX-2 inhibitory compound for which COX-2 in vitro IC₅₀ potency in said member is at least 30 fold greater, preferably at least 40 fold greater, more preferably at least 50 fold greater, more preferably still at least 60 fold greater, even more preferably still at least 80 fold greater, and most preferably at least 100 fold greater than its in vitro IC₅₀ potency against COX-1 in said member, wherein said inhibitor is a member selected from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones.

The present invention still further provides in another preferred aspect of the above-described method of treating or preventing pain and inflammatory processes and diseases in a member of the species Canis familiaris in need of such treatment, administration to said member of an amount therapeutically effective for treating pain and inflammation, of an anti-inflammatory selective COX-2 inhibitory compound which selectively inhibits substantially only inducible COX-2, with substantially no inhibition of corresponding constitutive COX-1.

There is further provided the above-described methods wherein said therapeutically effective amount of an anti-inflammatory compound of Formula (I) as defined, and especially of said (+)(S)-enantiomer of 6-chloro-α-methyl-9H-carbazole-2-acetic acid, is administered systemically to said member of Canis familiaris, wherein said systemic administration comprises: (1) injection or infusion into suitable body tissues or cavities of a pharmaceutical composition containing said inhibitor in suitable liquid form for delivering said inhibitor by systemic administration which is intraarterial, intra- or transdermal, subcutaneous, intramuscular, intraspinal, intrathecal, or intravenous, where said inhibitor is: (a) contained in solution as a solute; (b) contained in the discontinuous phase of an emulsion, or the discontinuous phase of an inverse emulsion which inverts upon injection or infusion, said emulsions containing suitable emulsifying agents; or (c) contained in a suspension as a suspended solid in colloidal or microparticulate form, said suspension containing suitable suspending agents; (2) injection or infusion into suitable body tissues or cavities of a pharmaceutical composition containing said inhibitor in suitable liquid form to serve as a depot for delivering said inhibitor by systemic administration, wherein said composition provides storage of said inhibitor and thereafter delayed-, sustained-, and/or controlled-release of said inhibitor for systemic distribution; (3) instillation, inhalation or insufflation into suitable body tissues or cavities of a pharmaceutical composition containing said inhibitor in suitable solid form for delivering said inhibitor, where said inhibitor is: (a) contained in a solid implant composition which is installed in suitable body tissues or cavities, said composition providing delayed-, sustained-, and/or controlled-release of said inhibitor; (b) contained in a particulate composition which is inhaled into the lungs; or (c) contained in a particulate composition which is blown into suitable body tissues or cavities, where said composition optionally provides delayed-, sustained-, and/or controlled-release of said inhibitor; or (4) ingestion of a pharmaceutical composition containing said inhibitor in suitable solid or liquid form for peroral delivery of said inhibitor, where said inhibitor is: (a) contained in a solid dosage form; or (b) contained in a liquid dosage form. Suppositories may be regarded as a special type of implant, since they comprise bases which are solid at room temperature but melt at body temperature, slowly releasing the active ingredient with which they are impregnated into the surrounding tissue of the body, where the active ingredient becomes absorbed and transported to effect systemic administration. Dosage forms which permit transdermal and transmucosal administration to achieve systemic delivery are also contemplated, especially including transdermal patch technology.

There is further provided the above-described method of treating or preventing pain and inflammation comprising ingestion or administration of a solid peroral dosage form selected from the group consisting of delayed-release oral tablet, capsule, caplet, lozenge, troche, and multiparticulates, enteric-coated tablets and capsules which prevent release and absorption in the stomach to facilitate delivery distal to the stomach of the dog, sustained-release oral tablets, capsules and microparticulates which provide systemic delivery of the active ingredient in a controlled manner over at least a 10-hour period, a fast-dissolving tablet, encapsulated solutions, an oral paste, a granular form incorporated in or to be incorporated in the food of the dog being treated, and a chewable form in which said active ingredient is consumed along with the palatable chew, or may alternatively be delivered by leaching from the body of the chew which is not consumed, during mastication by the dog being treated. Also included for use with the above-described dosage forms are microencapsulated formulations of the active ingredient, which may then be incorporated into a tablet, capsule, or other final formulation. Still further, there is provided said method comprising ingestion of a liquid peroral dosage form selected from the group consisting of a solution, suspension, emulsion, inverse emulsion, elixir, extract, tincture, and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

There is still further provided the above-described methods wherein said therapeutically effective amount of an anti-inflammatory compound of Formula (I) as defined, is administered locally to a site of inflammation in said member of Canis familiaris. There is still further provided said method of local administration wherein said local administration comprises: (1) injection or infusion into a local site of inflammation of a pharmaceutical composition containing said inhibitor in suitable liquid form for delivering said inhibitor by local administration which is intraarterial, intraarticular, intrachondrial, intracostal, intracystic, intra- or transdermal, intrafasicular, intraligamentous, intramedulary, intramuscular, intraneural, intraosteal, intrapelvic, intrapericardial, intraspinal, intrasternal, intrasynovial, intratarsal, or intrathecal; including components which provide delayed-release, controlled-release, and/or sustained-release of said inhibitor into said local site of inflammation; where said inhibitor is contained: (a) in solution as a solute; (b) in the discontinuous phase of an emulsion, or the discontinuous phase of an inverse emulsion which inverts upon injection or infusion, said emulsions containing suitable emulsifying agents; or (c) in a suspension as a suspended solid in colloidal or microparticulate form, said suspension containing suitable suspending agents; (2) injection or infusion of a pharmaceutical composition containing said inhibitor in suitable liquid form to serve as a depot for delivering said inhibitor to said local site of inflammation; wherein said composition provides storage of said inhibitor and thereafter delayed-, sustained-, and/or controlled-release of said inhibitor into said local site of inflammation; or (3) instillation, inhalation or insufflation of a pharmaceutical composition containing said inhibitor in suitable solid form for delivering said inhibitor to said local site of inflammation, where said inhibitor is contained: (a) in a solid implant composition which is installed in said local site of inflammation, said composition optionally providing delayed-, sustained-, and/or controlled-release of said inhibitor to said local site of inflammation; (b) in a particulate composition which is inhaled into a local site of inflammation comprising the lungs; or (c) in a particulate composition which is blown into a local site of inflammation, where said composition optionally provides delayed-, sustained-, and/or controlled-release of said inhibitor to said local site of inflammation. Other specific dosage forms for local administration are also within the scope of the present invention. For example, compositions to be applied to the skin, preferably with enhancement of absorption by mechanical working of the composition into the skin as by rubbing, may be used to deliver the inhibitor active ingredient into a local area, such as an inflammed joint, in need of such treatment. Such compositions may be in the form of gels, lotions, balms, ointments, and other formulations designed for topical application.

There is still further provided the above-described methods wherein the therapeutically effective amount of anti-inflammatory inhibitor is administered to said member of the species Canis familiaris in an amount, expressed as mg per kg of body weight of said member-per day, ranging from about 0.01 mg/kg to about 20.0 mg/kg/day, preferably from about 0.1 mg/kg to about 12.0 mg/kg/day, more preferably from about 0.5 mg/kg to about 10.0 mg/kg/day, and most preferably from about 0.5 mg/kg to about 8.0 mg/kg/day. Administration of 6-chloro-α-methyl-9H-carbazole-2-acetic acid is typically provided by dosing at a rate of about 4.0 mg/kg/day.

There is additionally provided in accordance with the present invention a pharmaceutical composition for treating or preventing pain and inflammatory processes and diseases in a member of the species Canis familiaris in need of such treatment, comprising a pharmaceutically acceptable carrier together with an amount therapeutically effective for treating pain and inflammation, of an anti-inflammatory selective inhibitor of cyclo-oxygenase-2 (COX-2) wherein the selectivity ratio of COX-2 to COX-1 is at least 3:1 based on ex vivo inhibition levels in whole blood measured at a dose giving ≧80% COX-2 inhibition, and preferably ≧90% COX-2 inhibition. In a preferred aspect of said pharmaceutical composition for treating or preventing pain and inflammatory processes and diseases, said therapeutically effective anti-inflammatory selective COX-2 inhibitor has an in vitro IC₅₀ potency of at least 30 fold greater, preferably at least 40 fold greater, more preferably at least 50 fold greater, more preferably still at least 60 fold greater, even more preferably at least 80 fold greater, and most preferably at least 100 fold greater than cyclo-oxygenase-1 (COX-1) in vitro IC₅₀ potency; wherein said inhibitor is a member selected from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones.

There is further provided the above-described pharmaceutical composition wherein said inhibitor is a member selected from the group consisting of arylpropionic acids; and further still, said inhibitor is a compound of above-defined Formula (I). There is further provided the above-described pharmaceutical composition wherein the cyclb-oxygenase-2 (COX-2) in vitro IC₅₀ potency of the inhibitory compound of Formula (I) is at least 100 fold greater than the cyclo-oxygenase-1 (COX-1) in vitro IC₅₀ potency thereof; and wherein one of X and Y is H and the other is methyl; and wherein when both resulting enantiomers are present, (+)(S) enantiomer is present in amount of at least 75%. In particular, there is provided the above-described pharmaceutical composition wherein for Formula (I), for R² n=1, one of X and Y is H and the other is methyl, and A is hydroxy, (_{C}1-C₂) alkoxy, or amino; R⁶ is chloro or trifluoromethyl; and R⁹ is H, methyl, acetyl, benzoyl, or acetyloxy; and wherein when both resulting enantiomers are present together, (+)(S) enantiomer is present in amount of at least 85%, preferably at least 90%, more preferably at least 95%, and most preferably at least 99%.

There is still further provided the above-described pharmaceutical compositions in which said inhibitor comprises 6-chloro-α-methyl-9H-carbazole-2-acetic acid; and wherein when both resulting enantiomers are present together, (+)(S) enantiomer is present in an amount of at least 85%, preferably at least 90%, more preferably at least 95%, and most preferably at least 99%. In particular, there is provided the above- and below-described pharmaceutical composition in which said inhibitor is comprised entirely of (+)(S) enantiomer of 6-chloro-α-methyl-9H-carbazole-2-acetic acid.

There is also provided the above-described pharmaceutical compositions wherein the therapeutically effective amount of anti-inflammatory inhibitor present is sufficient, in the context of the dosage regimen and administration parameters employed, to provide a member being treated with an amount of said inhibitor, expressed as mg per kg of body weight of said member per day, ranging from about 0.01 mg/kg to about 20.0 mg/kg/day, preferably from about 0.1 mg/kg to about 12.0 mg/kg/day, more preferably from about 0.5 mg/kg to about 10.0 mg/kg/day, and most preferably from about 0.5 mg/kg to about 8.0 mg/kg/day. Administration of 6-chloro-α-methyl-9H-carbazole-2-acetic acid is typically provided by dosing at a rate of about 4.0 mg/kg/day.

In a still further preferred aspect of the pharmaceutical compositions of the present invention there is provided an anti-inflammatory selective COX-2 inhibitor which selectively inhibits substantially only inducible COX-2, with substantially no inhibition of corresponding constitutive COX-1. In particular a preferred embodiment thereof comprises a pharmaceutically acceptable carrier together with a therapeutically effective amount for treating pain and inflammation, of an anti-inflammatory selective inhibitor of COX-2 comprising a compound of Formula (I) wherein for R², n=1, one of X and Y is H, and the other is methyl, and A is hydroxy, (C₁-C₂) alkoxy, or amino; R⁶ is chloro or trifluoromethyl; and R⁹ is H, methyl, acetyl, benzoyl, or methoxycarbonyl; and (+)(S) enantiomer is present in amount of at least 99%. Especially, said inhibitor is comprised entirely of (+)(S)-enantiomer of 6-chloro-α-methyl-9H-carbazole-2-acetic acid. Preferably in said pharmaceutical composition, said therapeutically effective amount of said inhibitor is sufficient, in the context of the dosage regimen and administration parameters employed, to provide a member being treated with an amount of said inhibitor, expressed as mg per kg of body weight of said member per day, ranging from about 0.5 mg/kg/day to about 8.0 mg/kg/day.

There is still further provided the above-described pharmaceutical composition in a dosage form which can provide the required therapeutically effective amount for treating pain and inflammation, of anti-inflammatory selective COX-2 inhibitor in a convenient regimen. However, a number of the above- and below-described pharmaceutical compositions are intended to be long-acting, i.e., providing inhibitor activity for longer than just hours or a single day, and instead providing such activity for several days up to a week or more. The implants and depots in particular are examples of such long-acting pharmaceutical compositions, and some of these are intended to provide inhibitor activity for up to a month or more. The required therapeutically effective amount of inhibitor necessary to treat or prevent pain and inflammation, expressed as the mg per kg of body weight of said member of species Canis familiaris per day, ranges from about 0.01 mg/kg to about 20.0 mg/kg/day, preferably from about 0.1 mg/kg to about 12.0 mg/kg/day, more preferably from about 0.5 mg/kg to about 10.0 mg/kg/day, and most preferably from about 0.5 mg/kg to about 8.0 mg/kg/day. Administration of 6-chloro-α-methyl-9H-carbazole-2-acetic acid is typically provided by dosing at a rate of about 4.0 mg/kg/day.

In particular, there is further provided the above-described pharmaceutical compositions wherein said therapeutically effective amount for treating pain and inflammation, of an anti-inflammatory inhibitor, is provided in a dosage form suitable for systemic administration to said member of Canis familiaris, wherein said pharmaceutical composition contains said inhibitor in suitable liquid form for delivering said inhibitor by: (1) injection or infusion which is intraarterial, intra- or transdermal, subcutaneous, intramuscular, intraspinal, intrathecal, or intravenous, wherein said inhibitor: (a) is contained in solution as a solute; (b) is contained in the discontinuous phase of an emulsion, or the discontinuous phase of an inverse emulsion which inverts upon injection or infusion, said emulsions containing suitable emulsifying agents; or (c) is contained in a suspension as a suspended solid in colloidal or microparticulate form, said suspension containing suitable suspending agents; (2) injection or infusion into suitable body tissues or cavities as a depot, wherein said composition provides storage of said inhibitor and thereafter delayed-, sustained-, and/or controlled-release of said inhibitor for systemic distribution; (3) instillation, inhalation or insufflation into suitable body tissues or cavities of said pharmaceutical composition in suitable solid form, where said inhibitor: (a) is contained in a solid implant composition providing delayed-, sustained-, and/or controlled-release of said inhibitor; (b) is contained in a particulate composition to be inhaled into the lungs; or (c) is contained in a particulate composition to be blown into suitable body tissues or cavities, where said composition optionally provides delayed-, sustained-, and/or controlled-release of said inhibitor; or (4) ingestion of said pharmaceutical composition in suitable solid or liquid form for peroral delivery of said inhibitor, where said inhibitor: (a) is contained in a solid dosage form; or (b) is contained in a liquid dosage form.

In an especially preferred aspect of the above-described pharmaceutical compositions which provide delayed-, sustained-, and/or controlled-release of said anti-inflammatory selective COX-2 inhibitor, there is included all such dosage forms which produce >80% inhibition of COX-2 isozyme activity and result in a plasma concentration of said inhibitor of at least 10 pg/mL for at least 4 hrs; preferably for at least 8 hrs; more preferably for at least 12 hrs; more preferably still for at least 16 hrs; even more preferably still for at least 20 hrs; and most preferably for at least 24 hrs. Preferably, there is included the above-described dosage forms which produce >80% inhibition of COX-2 isozyme activity and result in a plasma concentration of said inhibitor of at least 15 lug/mL for at least 4 hrs, preferably for at least 8 hrs, more preferably for at least 12 hrs, still more preferably for at least 20 hrs, and most preferably for at least 24 hrs. More preferably, there is included the above-described dosage forms which produce ≧90% inhibition of COX-2 isozyme activity and result in a plasma concentration of said inhibitor of at least 20 μg/mL for at least 4 hrs, preferably for at least 8 hrs, more preferably for at least 12 hrs, still more preferably for at least 20 hrs, and most preferably for at least 24 hrs.

Particular dosage forms of the above-described pharmaceutical compositions include suppositories as a special type of implant, comprising bases which are solid at room temperature but melt at body temperature, slowly releasing the active ingredient with which they are impregnated into the surrounding tissue of the body, where the active ingredient becomes absorbed and transported to effect systemic administration; solid peroral dosage forms selected from the group consisting of delayed-release oral tablet, capsule, caplet, lozenge, troche, and multiparticulates, enteric-coated tablets and capsules which prevent release and absorption in the stomach to facilitate delivery distal to the stomach of the dog, sustained-release oral tablets, capsules and microparticulates which provide systemic delivery of the active ingredient in a controlled manner up to a 24-hour period, a fast-dissolving tablet, encapsulated solutions, an oral paste, a granular form incorporated in or to be incorporated in the food of the dog being treated, and a chewable form in which said inhibitor active ingredient is consumed along with the palatable chew, or may alternatively be delivered by leaching from the body of the chew which is not consumed, during mastication by the dog being treated; liquid peroral dosage forms selected from the group consisting of solutions, suspensions, emulsions, inverse emulsions, elixirs, extracts, tinctures, and concentrates, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

There is further provided the above-described pharmaceutical compositions wherein said therapeutically effective amount for treating pain and inflammation, of said anti-inflammatory inhibitor, is provided in a dosage form suitable for local administration to a site of inflammation in said member of Canis familiaris, wherein said pharmaceutical composition contains said inhibitor in suitable liquid form for delivering said inhibitor by: (1) injection or infusion into a local site of inflammation, which is intraarterial, intraarticular, intrachondrial, intracostal, intracystic, intra- or transdermal, intrafasicular, intraligamentous, intramedulary, intramuscular, intranasal, intraneural, intraocular, i.e., opthalmic administration, intraosteal, intrapelvic, intrapericardial, intraspinal, intrasternal, intrasynovial, intratarsal, or intrathecal; including components which provide delayed-release, controlled-release, and/or sustained-release of said inhibitor into said local site of inflammation; where said inhibitor is contained: (a) in solution as a solute; (b) in the discontinuous phase of an emulsion, or the discontinuous phase of an inverse emulsion which inverts upon injection or infusion, said emulsions containing suitable emulsifying agents; or (c) in a suspension as a suspended solid in colloidal or microparticulate form, said suspension containing suitable suspending agents; or (2) injection or infusion as a depot for delivering said inhibitor to said local site of inflammation; wherein said composition provides storage of said inhibitor and thereafter delayed-, sustained-, and/or controlled-release of said inhibitor into said local site of inflammation, and wherein said composition also includes components which ensure that said inhibitor has predominantly local activity, with little systemic carryover activity; or wherein said pharmaceutical composition contains said inhibitor in suitable solid form for delivering said inhibitor by: (3) instillation, inhalation or insufflation to said local site of inflammation, where said inhibitor is contained: (a) in a solid implant composition which is installed in said local site of inflammation, said composition optionally providing delayed-, sustained-, and/or controlled-release of said inhibitor to said local site of inflammation; (b) in a particulate composition which is inhaled into a local site of inflammation comprising the lungs; or (c) in a particulate composition which is blown into a local site of inflammation, where said composition includes components which will ensure that said inhibitor has predominantly local activity, with insignificant systemic carryover activity, and optionally provides delayed-, sustained-, and/or controlled-release of said inhibitor to said local site of inflammation.

There is provided in accordance with the present invention combinations of one or more other therapeutically active agents together with the active ingredients for treating pain and inflammation which make up the above-described pharmaceutical compositions of the present invention. Where a joint has become seriously inflammed and infected at the same time by microorganisms, e.g., bacteria, fungi, protozoa, virus and the like, the active ingredient of the present invention will desirably be administered in combination with one or more antibiotic, antifungal, antiprotozoal, antiviral or similar therapeutic agents. Further, the active ingredient of the present invention may be administered not only in combination with other NSAIDs, but in combination as well with inhibitors of other mediators of inflammation, comprising one or more members selected from the group consisting essentially of the classes of such inhibitors and examples thereof which include, H₁-receptor antagonists; kinin-B₁- and B₂-receptor antagonists; prostaglandin inhibitors such as PGD-, PGF-PGI₂ and PGE-receptor antagonists; thromboxane A₂ (TXA2-) inhibitors; 5- and 12-lipoxygenase inhibitors; leukotriene LTC₄-, LTD₄/LTE₄-, and LTB₄-inhibitors; PAF-receptor antagonists; gold in the form of an aurothio group together with various hydrophilic groups; immunosuppressive agents, e.g., cyclosporine, azathioprine, and methotrexate; anti-inflammatory glucocorticoids; penicillamine; hydroxychloroquine; anti-gout agents, e.g., colchicine, xanthine oxidase inhibitors, e.g., allopurinol, and uricosuric agents, e.g., probenecid, sulfinpyrazone, and benzbromarone. It is further provided that the anti-inflammatory agents of the present invention are administered in combination with therapeutic agents intended for the treatment of disease conditions, syndromes and symptoms found in older dogs, comprising one or more members selected from the group consisting essentially of the therapeutic agents and conditions being treated which include cognitive therapeutics to counteract memory loss and impairment; anti-hypertensives and other cardiovascular drugs intended to offset the consequences of atherosclerosis, including hypertension, myocardial ischemia including angina, congestive heart failure, and myocardial infarction, selected from diuretics, vasodilators such as hydralazine, β-adrenergic receptor antagonists such as propranolol, angiotensin-li converting enzyme inhibitors (ACE-inhibitors) such as enalapril used to treat geriatric dogs with mitral insufficiency, and enalapril alone and in combination with neutral endopeptidase inhibitors, angiotensin II receptor antagonists such as losartan, renin inhibitors, calcium channel blockers such as nifedipine, sympatholytic agents such as methyldopa, α₂-adrenergic agonists such as clonidine, α-adrenergic receptor antagonists such as prazosin, and HMG-CoA-reductase inhibitors (anti-hypercholesterolemics) such as lovastatin or atorvastatin; antineoplastic agents, especially antimitotic drugs including the vinca alkaloids such as vinblastine and vincristine; growth hormone secretagogues; strong analgesics; local and systemic anesthetics; and H₂-receptor antagonists and other gastroprotective agents. It is still further provided that the above combinations of therapeutic agents are used to treat acute conditions in dogs, including bacterial infections occurring simultaneously with degenerative joint disease; and to treat chronic conditions in dogs, wherein the regimen used for this purpose comprises administration of the anti-inflammatory agents of the present invention in combination with other medications used on a regularly scheduled basis for treating chronic conditions including osteoarthritis; formulation of the anti-inflammatory agents of the present invention with one or more other therapeutic agents which are to form the intended combination, into a convenient dosage form containing all of the drugs forming the combination, including wherein said different drugs have varying half-lives, by creating controlled-release forms of said drugs with different release times which achieves relatively uniform dosing; a medicated feed dosage form in which said drugs used in the combination are present together in admixture in said feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the dog being treated, even though the individual drugs making up said combination are not being administered to said dog simultaneously.

It is also contemplated that in accordance with the present invention there will also be provided a package suitable for use in commerce for the therapeutic treatment or prevention of pain and inflammation processes and diseases in a member of the species Canis familiaris in need of such treatment, comprising a suitable container which may be in the form of an outer package and an inner container removably housed therein; enclosed in said container a suitable dosage form of an active ingredient comprising a selective inhibitor of COX-2 wherein the selectivity ratio of COX-2 to COX-1 is at least 3:1 based on ex vivo inhibition levels in whole blood measured at a dose giving ≧80% COX-2 inhibition, preferably ≧90% COX-2 inhibition, as described elsewhere herein; and associated with said container printed instructional and informational material, which may be attached to said container, enclosed in said container, or displayed as an integral part of said container, said instructional and informational material stating in words which convey to a reader thereof that said active ingredient, when administered to a dog to be treated, effectively inhibits COX-2 induced at an existing or expected site of pain and inflammation in said dog, wherein the selectivity ratio of COX-2 to COX-1 is at least 3:1 based on ex vivo inhibition levels in whole blood measured at a dose giving ≧80% COX-2 inhibition, thereby treating or preventing said pain and inflammation which would otherwise result therefrom. In a preferred aspect of said package suitable for use in commerce, said instructional and informational material will state in words which convey to a reader thereof that said active ingredient when administered to a dog to be treated will provide effectively complete inhibition of induced COX-2 in said dog, thereby treating or preventing pain and inflammation therein; but also that when said ingredient is thus administered to said dog it will cause substantially no inhibition of constitutive COX-1 in said dog, whereby undesirable gastrointestinal and other adverse effects resulting from substantial inhibition of said COX-1 will be avoided in effectively most dogs.

It is also contemplated that in accordance with the present invention there will further be provided a package of the type described immediately above, comprising a suitable container as described; enclosed in said container an oral dosage form of a compound of Formula (I), which may be in the form of a chewable or ingestible oral tablet, a unit dose packet referred to as a sachet, a suspension made from a unit dose packet, a powder for oral suspension, or an oral suspension per se, which does not exhibit an adverse food effect; and associated with said container printed instructional and informational material as described, which is free of any express or implied limitation with respect to whether said oral dosage form can be taken with or without food.

### DETAILED DESCRIPTION OF THE INVENTION

The object of the present invention is to find a solution to a serious problem which has confronted the veterinary field for decades with regard to the need for an effective but safe anti-inflammatory treatment for dogs. The seriousness and intractability of this problem arises from the fact that virtually all anti-inflammatory agents, especially the NSAIDs, which have been tested for use in dogs, have had altogether unacceptable and sometimes dangerous adverse reactions in dogs which have greatly curtailed their use. By far the most wide-spread and threatening of these adverse reactions is disturbance and irritation of the gastrointestinal mucosa leading to ulceration, hemorrhage and eventually perforation and peritonitis. These undesirable adverse reactions are mediated by the inhibition of various prostaglandins by the NSAID inhibitors, resulting in a restricted blood supply to the protective gastrointestinal muscosa, which in turn is seriously diminished both in total mass and in protective functioning.

Gastric damage by NSAIDs is brought about by at least two distinct mechanisms. Local irritation by orally administered NSAIDs permits back diffusion of acid into the gastric mucosa, inducing tissue damage. Parenteral administration, on the other hand, can also cause damage and bleeding, which has been correlated with inhibition of the biosynthesis of gastric prostaglandins that serve a cytoprotective function in the gastric mucosa. These prostaglandins, especially PGI₂ and PGE₂, inhibit acid secretion by the stomach, increase mucosal blood flow, and promote the secretion of cytoprotective mucus in the intestine. Other undesirable side effects of the NSAIDs include disturbances in platelet function, the prolongation of gestation or spontaneous labor, changes in renal function, and hypersensitivity reactions.

All of the above-described undesirable side effects of NSAIDs probably depend upon blockade of the synthesis of endogenous prostaglandins. Accordingly, there is significant interest in the discovery of NSAIDs which do not result in the systemic production of such undesirable side effects. Dogs are not only especially vulnerable to inflammatory processes and diseases, such as degenerative joint disease, but they are also particularly susceptible to complications from the adverse gastrointestinal reactions which ensue.

As used herein, the term “dog(s)” denotes any member of the species Canis familiaris, of which there are a large number of different breeds. While laboratory determinations of biological activity may have been carried out using a particular breed, it is contemplated that the inhibitory compounds of the present invention will be found to be useful for treating pain and inflammation in any of these numerous breeds.

In its broadest aspects, the gist of the present invention is the surprising discovery that a small genus of anti-inflammatory agents, of which carprofen, 6-chloro-α-methyl-9H-carbazole-2-acetic acid, is the best example, possesses a high degree of canine cyclo-oxygenase-2 (COX-2) selectivity, and that this selectivity is unique among the large class of carboxyl- and carboxy(C₁-C₄)alkyl aryl and/or heteroaryl anti-inflammatory agents to which the carprofen genus of compounds also belongs. This unique and unexpected selectivity in dogs has far-reaching implications for the safe and effective treatment of dogs suffering from any one of a number of inflammatory processes and diseases.

It is now reasonably well accepted in the art of treating inflammatory processes and diseases, especially in dogs, that the cascade of endogenous reactions which produces various prostaglandin compounds in the body, beginning with arachidonic acid, is carried forward by an essential enzyme called cyclo-oxygenase. It has been established that this enzyme exists in two isozyme forms which are separate and act independently, a constitutive cyclo-oxygenase-1 (COX-1) isozyme and a cyclo-oxygenase-2 (COX-2) isozyme. The COX-1 isozyme in dogs is responsible for producing prostaglandins which perform important functions in the stomach, intestine, kidney and blood platelets, some of which are protective in nature, especially with respect to the gastrointestinal mucosa. The COX-2 isozyme in dogs is responsible for producing prostaglandins such as PGE₂ which mediate acute and chronic inflammation within neutrophils, macrophages, endothelial cells and fibroblasts, in which the COX-2 gene is expressed. COX-2 can be induced by endotoxin, lipopolysaccharide (LPS), various cytokines, e.g., IL-1 and TNF, growth factors, e.g., EGF and PDGF, and many other agents. For example, the COX-2 isozyme can be detected by immunoblotting in mononuclear cells of the pleural exudate after carrageenan-induced pleurisy in a rat model.

Ultimately, the objective in the art has been to discover compounds which are able to inhibit the activity of the COX-2 isozyme in dogs on effectively a 100% basis, while at the same time inhibiting the activity of the COX-1 isozyme on effectively a 0% basis. As a practical matter, this has amounted to a search for compounds which selectively inhibit the COX-2 isozyme, i.e., compounds which provide optimal anti-inflammatory therapy through relatively high levels of COX-2 isozyme inhibition at a dose which also produces minimal undesirable side effects through relatively low levels of COX-1 isozyme inhibition. Until the discovery of the present invention described herein, the only compounds which have been shown to exhibit COX-2 selectivity are those having a sulfonyl or sulfonamido group, rather than a carboxyl group, as is characteristic of the vast majority of classical NSAIDs. These observations have led to speculation that the Arg 150 residue of the COX-1 isozyme is not an essential binding site for selective inhibitors of COX-2, whereas the Arg 150 residue is essential for COX-1 activity because it binds the terminal carboxyl group of arachidonic acid and analogously, the carboxylic acid function of the above-referred to classical NSAIDs. It would be expected that COX-2 selective inhibitors would bind to some unique feature of the canine COX-2 isozyme, rather than to a feature that was common to the canine COX-1 and COX-2 isozymes.

Accordingly, the present invention provides a method of selectively inhibiting inducible cyclo-oxygenase-2 (COX-2) with respect to inhibition of corresponding constitutive cyclo-oxygenase-1 (COX-1), and thereby treating or preventing pain and inflammatory processes and diseases associated therewith in a member of the species Canis familiaris in need of such treatment, wherein the selectivity ratio of COX-2 to COX-1 is at least 3:1 based on in vivo and ex vivo inhibition levels in whole blood measured at the dose or range of doses giving ≧90% COX-2 inhibition, comprising administering to said member an amount therapeutically effective for treating pain and inflammation in accordance with the above-recited limitations, of an anti-inflammatory compound.

The present invention further provides the above-described method of treating or preventing pain and inflammatory processes and diseases in a member of the species Canis familiaris wherein said anti-inflammatory selective COX-2 inhibitor comprises a compound of the formula:

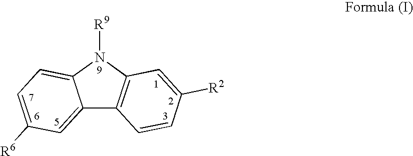

wherein:

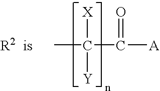

where A is hydroxy, (C₁-C₄)alkoxy, amino, hydroxy-amino, mono-(C₁-C₂)alkylamino, di-(C₁-C₂)alkylamino; X and Y are independently H or (C₁-C₂)alkyl; and n is 1 or 2;

R⁶ is halogen, (C₁-C₃)alkyl, trifluoromethyl, or nitro;

R⁹ is H; (C₁-C₂)alkyl; phenyl or phenyl-(C₁-C₂)alkyl, where phenyl is optionally mono-substituted by fluoro or chloro; —C(═O)—R, where R is (C₁-C₂)alkyl or phenyl, optionally mono-substituted by fluoro or chloro; or —C(═O)—O—R¹, where R¹ is (C₁-C₂)alkyl;

where X and Y are different, the (−)(R) and (+)(S) enantiomers thereof; and all pharmaceutically acceptable salt forms, prodrugs and metabolites thereof which are therapeutically active for treating or preventing pain and inflammation. Where the inhibitor of Formula (I) exists as (−)(R) and (+)(S) enantiomers, in accordance with the present invention there is provided the (+)(S) enantiomer alone, or where both enantiomers are present together, there is provided a racemic or a non-racemic mixture thereof.

It is quite surprising that the preferred carprofen genus of compounds, characterized by an α-methyl-acetic acid functionality, has many times greater canine COX-2 selectivity than any of the carboxyl-containing classical NSAIDs, as well as many times greater canine COX-2 selectivity than some of the sulfonyl and sulfonamide-containing NSAIDs accepted in the art as being highly COX-2 selective compounds. This aspect of the present invention is embodied in a method of treating or preventing inflammatory processes and diseases in a dog comprising administering to them an anti-inflammatory therapeutically effective amount of an inhibitor of canine cyclo-oxygenase-2 (COX-2) for which in vitro IC₅₀ potency in said dog is at least 50 fold greater than canine cyclo-oxygenase-1 (COX-1) in vitro IC₅₀ potency in said dog; wherein said inhibitor is a member selected from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones; and in particular is a member selected from the group consisting of carboxyl- and carboxy(C₁-C₄)alkyl aryl and/or heteroaryl anti-inflammatory agents. In particular, said inhibitor is a member selected from the group consisting of the aryl propionic acid class of non-steroidal anti-inflammatory drugs.

Carprofen and the genus of carprofen derivatives preferably utilized in the methods and compositions of the present invention may be prepared in accordance with methods of synthesis well known to the organic chemist of ordinary skill. For example, compounds of Formula (I) where R⁶ is halogen, (C₁-C₃)alkyl, trifluoromethyl, or nitro; and where R⁹ is H or methyl; may be prepared by reacting (1) a phenylhydrazine in which the phenyl portion has the desired R⁶ substitution and the α-nitrogen of the hydrazine has the desired R⁹ substitution; with (2) a cyclohexanone having the desired R² substitution. The resulting 1,2,3,4-tetrahydrocarbazole is then aromatized to produce the desired carbazole of Formula (I). The aromatization may be carried out using (1) an aromatizing agent, e.g., p-chloranil, o-chloranil, 2,3-dichloro-5,6-dicyanobenzoquinone (DDQ), sulfur, palladium on carbon, or lead oxide; in the presence of (2) a solvent such as xylene, benzene, toluene, quinoline, dimethylsulfoxide (DMSO), and dimethylformamide (DMF); (3) at a temperature in the range from room temperature to reflux-of the reaction mixture, preferably the latter.

Compounds of Formula (I) which are acids, i.e., where A is hydroxy, and salts of such acids with bases, can be converted to amides of Formula (I) where A is amino, hydroxyamino, mono-(C₁-C₂)alkylamino, and di-(C₁-C₂)alkylamino; by (1) forming the corresponding acid chloride by treatment with phosphorus pentachloride (PCI₅); followed by (2) reaction with the appropriate amine reactant to form the desired amide, carried out in the presence of an equivalent of pyridine or triethylamine to serve as the base for the proton transfer step and thereby remove the H⁺Cl⁻ which is formed. The same acid chlorides which are formed in step (1) can be reacted with the appropriate alkanol to provide the esters of Formula (I) where A Is (C₁-C₄)alkoxy. This reaction is also desirably carried out in the presence of a base such as pyridine which can neutralize the H⁺Cl⁻ being formed so that it does not interfere with any acid sensitive alkanol reactant.

The above-described synthetic approaches to preparation of the preferred carprofen genus of compounds utilized in the methods and compositions of the present invention are described in detail in U.S. Pat. No. 3,896,145, which is incorporated herein by reference in its entirety.

When “X” and “Y” are different in the definition of the “R²” substituent, then a chiral (asymmetric) carbon atom exists. A racemic mixture of (R)- and (S)-enantiomers results when there is a 50:50 mixture of the two enantiomers. In accordance with the present invention it has been discovered that the (S)-enantiomer of the preferred carprofen genus of compounds of Formula (I) having a chiral carbon is the enantiomer which possesses the surprising degree of unexpected canine COX-2 selectivity, a unique biological activity not possessed by virtually any of the classical NSAIDs having a carboxylic acid moiety, and especially not possessed by their S-enantiomers. Therefore, the (S)-enantiomer of the preferred carprofen genus of compounds of Formula (I) having a chiral carbon would possess a surprisingly reduced level of adverse gastrointestinal and other reactions in dogs compared to that of virtually all other NSAIDs having a carboxylic acid moiety, and especially compared to their S-enantiomers. Thus, it would also be wholly unexpected that the S-enantiomer of carprofen would have, by reason of its being a very highly selective inhibitor of the canine COX-2 isozyme, a surprisingly improved level of anti-inflammatory, analgesic and anti-pyretic activity in dogs compared to that of virtually all other NSAIDs characterized by a carboxylic acid moiety.

One especially preferred embodiment of the present invention is to use only the (S)-enantiomer of carprofen, 6-chloro-α-methyl-9H-carbazole-2-acetic acid, as the active ingredient or treating agent in the methods and compositions of the present invention. However, other embodiments are contemplated to be within the scope of this preferred genus of the present invention as well. For example, non-racemic mixtures of the (R)- and (S)-enantiomers can be used, and in that event the (S)-enantiomer is present in amount of at least 85%, preferably at least 90%, more preferably at least 95%, and most preferably at least 99%. Since the (R)- and (S)-enantiomers are identical in molecular weight, density, etc., it is unnecessary to state any basis for the above-recited percentages. In other words, they could be percentages by weight, volume, chemical equivalency, etc. The reason for including the above-indicated amounts of the (R)-enantiomer may be as simple as the practicalities of not being required to remove absolutely every last trace of the (R)-enantiomer from the racemic mixture. There can also be reasons for doing so which relate to beneficial overall biological properties.

It will also be appreciated by those in the art that the ranges of dosage amounts recited elsewhere herein for the preferred genus of carprofen compounds are being described with respect to a 50:50 racemic mixture of enantiomers, where a chiral compound is involved. This has been done largely as a matter of convenience. Where the active ingredient being used as a therapeutic agent comprises a mixture of enantiomers different from a 50:50 mixture, or where the therapeutic agent comprises substantially 100% of the (+)(S) or (−)(R) enantiomer alone, the person of ordinary skill in this art will be able to calculate the actual amount of dosage required in a very straightforward manner, simply by multiplying the dosage amounts recited by a factor which reflects the ratio of the amount of enantiomer being used to the amount present for the recited dosage based on a 50:50 mixture of the enantiomers. Accordingly, where the recited dosage is 4 mg/kg/day for the 50:50 racemic mixture, the corresponding dosage amount when substantially 100% of (+)(S) enantiomer is used one-half of the recited amount, i.e., 2 mg/kg/day.

Since the pharmaceutical compositions of the present invention containing a member of the preferred genus of carprofen compounds contemplate the use of racemic mixtures containing 50% of (S)-enantiomer, as well as non-racemic mixtures of about 99% or less of the (S)-enantiomer along with less than 50% of the (R)-enantiomer, resolution of racemates of the carprofen genus of compounds of Formula (I) having a chiral carbon into the optically active isomers must be carried out. This can be readily accomplished using known procedures and techniques inithe art. For example, some racemic mixtures can be precipitated as eutectics after which they can be separated. However, it is usually preferred to use chemical procedures for resolution, in accordance with which diastereomers are formed from the racemic mixture with an optically active resolving agent. For example, an optically active base, e.g., D-α-methylbenzylamine, which can be reacted with the carboxyl group. The diastereomers thus formed are then separated by selective crystallization and converted to the corresponding optical isomer.

Included within the scope of the present invention are all of the anti-inflammatory therapeutically active and pharmaceutically acceptable salt forms, prodrugs and metabolites of the preferred carprofen genus of compounds used in the present invention. This especially includes acid addition salts thereof, where “A” is defined as anything other than “hydroxy”, formed by treating the compounds of Formula (I) with pharmaceutically acceptable organic and inorganic acids, e.g., hydrohalides such as hydrochloride, hydrobromide, hydroiodide; other mineral acids and their corresponding salts such as sulfate, nitrate, phosphate, etc.; and alkyl- and mono-arylsulfonates such as ethanesulfonate, toluenesulfonate, and benzenesulfonate; and other organic acids and their corresponding salts such as acetate, tartrate, maleate, succinate, citrate, benzoate, salicylate, ascorbate, etc.

Where “A” is defined as “hydroxy” in the preferred carprofen genus of compounds used in the present invention, salts thereof may be formed by treatment with pharmaceutically acceptable bases. Examples of such bases are alkali metal hydroxides including potassium hydroxide, sodium hydroxide, and lithium hydroxide; alkaline earth metal hydroxides such as barium hydroxide and calcium hydroxide; alkali metal alkoxides, e.g., potassium ethanolate and sodium propanolate; and various organic bases such as piperidine, diethanolamine, and N-methylglutamine. Also included are the aluminum salts of the compounds of Formula (I).

In addition to the use of the various above-described salt forms of the compounds of Formula (I), there is included within the scope of the present invention the use as active ingredients of all analgesic and anti-inflammatory therapeutically active and pharmaceutically acceptable prodrugs and metabolites of the above-recited compounds. In particular, this includes those derivatives where R⁹ is defined as (C₁-C₂)alkyl, especially methyl; phenyl or phenyl-(C₁-C₂)alkyl, especially benzyl, where phenyl is optionally mono-substituted by fluoro or chloro, especially 4-fluoro-phenyl; —C(═O)—R, where R is (C₁-C₂)alkyl or phenyl, especially acetyl and benzoyl, where phenyl is optionally mono-substituted by fluoro or chloro; or —C(═O)—O—R¹, where R¹ is (C₁-C₂)alkyl, especially acetyloxy. These N-moieties are readily cleaved during metabolism of the compound of Formula (I), making these particular derivatives desirable prodrugs.

The present invention has been described in the paragraphs immediately above particularly with regard to the preferred genus of carprofen compounds of Formula (I). However, the present invention contemplates a wider scope with regard to providing anti-inflammatory selective COX-2 inhibitors. As already indicated, such selective COX-2 inhibitors comprise those wherein the selectivity ratio of COX-2 to COX-1 is at least 3:1 based on in vivo and ex vivo inhibition levels in whole blood measured at the dose or range of doses giving ≧80% COX-2 inhibition. In accordance with pharmacokinetic (PK) data described further below, it has been demonstrated that a relationship exists between the pharmacological or toxic response in dogs to the anti-inflammatory selective COX-2 inhibitors of the present invention and the accessible concentration of said inhibitors, e.g., in the blood or plasma of said dogs. For purposes of the instant exposition of the present invention, said pharmacological response in dogs is regarded herein as the anti-inflammatory response resulting from inhibition of the COX-2 isozyme; while said toxic response in dogs is regarded herein as the undesirable side effect, e.g., gastrointestinal, response resulting from inhibition of the COX-1 isozyme. Thus, the concentration of inhibitory drug in the systemic circulation of said dogs will be related to the concentration of inhibitory drug at its sites of action.

As is well known, the most important pharmacokinetic parameters are bioavailability, the fraction of said drug which is absorbed into the systemic circulation; volume of distribution, relating to the amount of space in the body which is available to contain said drug; and clearance, relating to the body's ability to eliminate a particular drug. In accordance with the present invention, the selectivity ratio for COX-2 to COX-1, which must be at least 3:1, is determined on the basis of ex vivo measurement of the percentage (%) inhibition of each of said isozymes in canine whole blood. The procedures for making said ex vivo measurements may be briefly summarized as consisting of first administering the selective inhibitor test compound at a predetermined total dose level, e.g., 2 mg/lb to preselected dogs. The dose is administered in accordance with a predetermined dosing regimen, e.g., once a day (s.i.d.), twice a day (b.i.d.), etc., after which whole blood samples are collected from each test animal. Assays for COX-1 and COX-2 activity are based on stimulation of said blood with either a calcium ionophore for thromboxane B₂ (COX-1 activity) or lipopolysaccharide (LPS) for prostaglandin E₂ (COX-2 activity), respectively. These procedures are described in more detail in the further below exemplary descriptions.

COX-2 selectivity is an essential feature of the anti-inflammatory inhibitors useful in the present invention, and is required to be in a ratio of at least 3:1 compared to inhibition of COX-1 activity. It is insufficient for a given compound simply to possess a 3:1 selective ratio, since a given compound might possess the required selective ratio at some point over its total dosage range, but still fail to, provide adequate inhibition of COX-2 activity overall. Accordingly, it is additionally required that for an anti-inflammatory inhibitor to be within the scope of the present invention it must also provide ≧80% COX-2 activity inhibition, and preferably ≧90% COX-2 activity inhibition. This is deemed to be the minimum level of inhibition adequate to provide satisfactory pharmacological activity in terms of treating and preventing pain and inflammation.

It is further insufficient for a given compound simply to possess a 3:1 selective ratio at a given dose and to provide ≧80% COX-2 activity inhibition, preferably ≧90% COX-2 activity inhibition at some other dose. But rather, both criteria must be met at some level of exposure and at the same time, post-dose. Accordingly, it is also required that the 3:1 selective ratio occur at the dosage concentration or range of dosage concentrations which also provides ≧80% COX-2 activity inhibition.

In order to demonstrate the manner in which the above-described limitations would be applied to a potential selective COX-2 inhibitor, the following example is to be considered. Said candidate is evaluated in accordance with the above-described procedures for measuring COX-1 and COX-2 activity inhibition in whole blood samples at a total dose of 2 mg/lb administered s.i.d., with activity levels being determined at 2, 4 and 12 hours after administration. When data points for COX-1, COX-2 and untreated samples are plotted on a graph of % inhibition vs. time, it is found that the COX-1 curve drops fairly steeply from a %, inhibition at 2 hrs of 10-15% to no observed inhibition at 12 hrs. The COX-2 curve, on the other hand, is found to drop only slightly during this same period of time, declining from a % inhibition at 2 hrs of about 95% to a % inhibition at 12 hrs of about 90%.

Accordingly, it should be noted that at the dose of 2 mg/lb administered s.i.d. the potential inhibitor has satisfied (1) the at least 3:1 selectivity ratio, and (2) the ≧80% COX-2 inhibition limitations required for it to be an anti-inflammatory selective COX-2 inhibitor falling within the scope of the present invention. Because the data from both of these graphs has been compared over the same dose (2 mg/lb) and the same time period of 12 hrs, the third limitation has also been satisfied, namely that the first two limitations be satisfied at the same dose or range of doses.

Similar determinations can be made and graphs plotted of the resulting data for a number of different dosage amounts of said candidate compound. However, the combined results are demonstrated by means of a graph which plots % inhibition of COX-1 and COX-2 vs. dose or plasma concentration, rather than time. The curve representing the COX-2 data appears first and rises steeply, starting at the least dosage concentration which produces an identifiable % inhibition, and as the dosage approaches higher concentrations, reaching inhibition levels ≧80% and becoming asymptotic thereafter. The curve representing the COX-1 data appears later and rises less steeply than the curve of COX-2 data, becoming asymptotic at a significantly lower level of % inhibition as well. A candidate compound falls within the scope of the present invention if a line parallel to the y-axis and intercepting both curves at some dosage concentration results in data points which satisfy both the 3:1 ratio and ≧80% inhibition limitations.

The scope of the present invention with respect to those anti-inflammatory selective COX-2 inhibitors which are included therein can be viewed or expressed in different, although essentially equivalent terms to those above-used. For example, the present invention may be considered to provide particularly a method of treating or preventing pain and inflammatory processes and diseases in a member of the species Canis familiaris in need of such treatment, which comprises administering to said member a therapeutically effective amount for treating pain and inflammation, of an anti-inflammatory inhibitor of cyclo-oxygenase-2 (COX-2) for which in vitro IC₅₀ potency in said member is at least 30 fold greater, preferably at least 40 fold greater, more preferably at least 50 fold greater, more preferably still at least 60 fold greater, even more preferably at least 80 fold greater, and most preferably at least 100 fold greater than cyclo-oxygenase-1 (COX-1) in vitro IC₅₀ potency in said member; wherein said inhibitor is a member selected from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones.

In accordance with the above-described methods of the present invention, there is more particularly provided the above-described method of treating or preventing pain and inflammatory processes and diseases wherein said cyclo-oxygenase-2 (COX-2) in vitro IC₅₀ potency of the inhibitory compound of Formula (I) is at least 100 fold greater than said cyclo-oxygenase-1 (COX-1) in vitro IC₅₀ potency thereof; wherein one of X and Y is H and the other is methyl; and wherein when both resulting enantiomers are present together, (+)(S) enantiomer is present in amount of at least 75%. In particular, there is provided the above-described method wherein for Formula (I), for R², n=1, one of X and Y is H and the other is methyl, and A is hydroxy, (C₁-C₂)alkoxy, or amino; R⁶ is halo, especially chloro, or trifluoromethyl; and R⁹ is H, methyl, acetyl, benzoyl, or methoxycarbonyl; and wherein when both resulting enantiomers are present together, (+)(S) enantiomer is present in amount of at least 85%, preferably at least 90%, more preferably at least 95%, and most preferably at least 99%.

There is still further provided the above-described methods in which said inhibitor comprises 6-chloro-α-methyl-9H-carbazole-2-acetic acid; and wherein when both resulting enantiomers are present together, (+)(S) enantiomer is present in amount of at least 85%, preferably at least 90%, more preferably at least 95%, and most preferably at least 99%. In particular, there is provided the above-described method in which said inhibitor is comprised entirely of (+)(S)-enantiomer of 6-chloro-α-methyl-9H-carbazole-2-acetic acid.

The present invention may also be described as providing a method of selectively inhibiting substantially only inducible cyclo-oxygenase-2 (COX-2), with substantially no inhibition of corresponding constitutive cyclo-oxygenase-1 (COX-1), and thereby treating or preventing pain and inflammatory processes and diseases associated therewith in a member of the species Canis familiaris in need of such treatment, comprising administering to said member a therapeutically effective amount for treating pain and inflammation, of an anti-inflammatory compound of Formula (I) above where R², X, Y, n, A, R⁶, and R⁹ are as defined; including the (−)(R) and (+)(S) enantiomers; and all anti-inflammatory therapeutically active and pharmaceutically acceptable salt forms, prodrugs and metabolites of the above-recited compounds. Here again, where the inhibitor of Formula (I) exists as (−)(R) and (+)(S) enantiomers, in accordance with the present invention there is provided the (+)(S) enantiomer alone, or where both enantiomers are present together, there is provided a racemic or a non-racemic mixture thereof.

In a manner essentially parallel to the other above-descriptions thereof, the present invention may be further described as including the above-recited method of selectively inhibiting substantially only inducible cyclo-oxygenase-2 (COX-2), wherein said inhibitor comprises 6-chloro-α-methyl-9H-carbazole-2-acetic acid; and wherein when both resulting enantiomers are present, (+)(S) enantiomer is present in amount of at least 85%, preferably at least 90%, more preferably at least 95%, and most preferably at least 99%. In particular, there is provided the above-described method in which said inhibitor is comprised entirely of (+)(S)-enantiomer of 6-chloro-α-methyl-9H-carbazole-2-acetic acid.

When the compounds of Formula (I), or their enantiomers or salts, are to be used as active ingredients in the methods and compositions of the present invention, they can be incorporated into standard pharmaceutical dosage forms. For example, they are useful when administered in systemic or local, oral or parenteral applications and for this purpose are combined with the usual pharmaceutical excipients, diluents and adjuvants, e.g., organic and inorganic inert carrier materials such as water, gelatin, lactose, starch, magnesium stearate, talc, vegetable oils, gums, polyalkyleneglycols, etc. These pharmaceutical preparations can be employed in a solid form, e.g., as tablets, troches, suppositories, capsules, and especially in combination with or for admixture with a palatable food item suitable for dogs; or they can be administered in liquid form, e.g., as solutions, suspensions, standard and inverse emulsions, and elixirs. Pharmaceutical excipients and adjuvants which can be added include preservatives, antioxidants, antimicrobial agents and other stabilizers; wetting, emulsifying, and suspending agents, and anticaking compounds; fragrance and coloring additives; compositions for improving compressibility, or to create a delayed-, sustained-, or controlled-release of the active ingredient; and various salts to change the osmotic pressure of the pharmaceutical preparation or to act as buffers. Particular dosage forms which have been used with success include a 5% mixed-micelle solution of carprofen for intravenous injection, a 3% palatable paste, and oral tablets in 25 mg, 75 mg, and 100 mg dosages.

In the methods and compositions of the present invention, especially those wherein the inhibitor comprises 6-chloro-α-methyl-9H-carbazole-2-acetic acid and both resulting enantiomers are present together, it is a preferred embodiment to use a non-racemic mixture. Particularly, in such preferred non-racemic mixtures, it is desirable to have the (+)(S) enantiomer present in amount of at least 85%, preferably at least 90%, more preferably at least 95%, and most preferably at least 99%. Thus, in such non-racemic mixtures the (+)(S) enantiomer will be the predominant component, not only because it is significantly more potent than the (−)(R) enantiomer in inhibiting cyclo-oxygenase-2 (COX-2), but also because it is highly selective with respect to inhibiting cyclo-oxygenase-2 (COX-2) as compared to cyclo-oxygenase-1 (COX-1). The correspondingly smaller amounts of the (−)(R) enantiomer, i.e., less than 15%, less than 10% and less than 5%, respectively, are optionally included where a balance of cyclo-oxygenase or other enzyme inhibitory properties is deemed desirable. Where the amount of (−)(R) enantiomer present is less than 5% and less than 1%, the reason for the inclusion will usually reflect the practicalities of the method used to resolve the enantiomers. Where this method is time consuming or demanding of resources, it will often be desirable, from a practical standpoint, to simply allow this smaller proportion of the (−)(R) enantiomer to be carried over into the final, non-racemic mixture final product.

The anti-inflammatory inhibitors of Formula (I) of the present invention may be administered systemically to a dog to be treated as a pharmaceutical composition in suitable liquid form by injection or infusion. There are a number of sites and organ systems in the body of the dog which will allow the properly formulated pharmaceutical composition, once injected or infused, to permeate the entire body and all of the organ system of the dog being treated. An injection is a single dose of the pharmaceutical composition forced, usually by a syringe, into the tissue involved. The most common types of injections are intramuscular, intravenous, and subcutaneous. By contrast, an infusion is the gradual introduction of the pharmaceutical composition into the tissue involved. The most common type of infusion is intravenous. Other types of injection or infusion comprise intraarterial, intra- or transdermal (including subcutaneous), or intraspinal especially intrathecal. In these liquid pharmaceutical compositions, the anti-inflammatory inhibitor may be contained in solution as the solute. This is the most common and most preferred type of such composition, but requires an inhibitor in a salt form that has reasonably good aqueous solubility. Water. (or saline) is by far the most preferred solvent for such compositions. Occasionally supersaturated solutions may be utilized, but these present stability problems that make them impractical for use on an everyday basis.

If it is not possible to obtain a form of some compound of Formula (I) that has the requisite degree of aqueous solubility, as may sometimes occur, it is within the skill of the artisan to prepare an emulsion, which is a dispersion of small globules of one liquid, the discontinuous or internal phase, throughout a second liquid, the continuous or external phase, with which it is immiscible. The two liquids are maintained in an emulsified state by the use of emulsifiers which are pharmaceutically acceptable. Thus, if the anti-inflammatory inhibitor is a water-insoluble oil, it can be administered in an emulsion of which it is the discontinuous phase. Also where the inhibitor is water-insoluble but can be dissolved in a solvent which is immiscible with water, an emulsion can be used. While the inhibitor would most commonly be used as the discontinuous or internal phase of what is referred to as an oil-in-water emulsion, it could also be used as the discontinuous or internal phase of an inverse emulsion, which is commonly referred to as a water-in-oil emulsion. Here the anti-inflammatory inhibitor is soluble in water and could be administered as a simple aqueous solution. However, inverse emulsions invert upon injection or infusion into an aqueous medium such as the blood, and offer the advantage of providing a more rapid and efficient dispersion of the inhibitor into that aqueous medium than can be obtained using an aqueous solution. Inverse emulsions are prepared by using suitable, pharmaceutically acceptable emulsifying agents well known in the art. Where the anti-inflammatory inhibitor has limited water solubility, it may also be administered as a suspended solid in colloidal or microparticulate form in a suspension prepared using suitable, pharmaceutically acceptable suspending agents. The suspended solids containing the inhibitor may also be formulated as delayed-, sustained-, and/or controlled-release compositions.

While systemic administration will most frequently be carried out by injection or infusion of a liquid, there are many situations in which it will be advantageous or even necessary to deliver the anti-inflammatory inhibitor as a solid. Systemic administration of solids is carried out by instillation, inhalation or insufflation of a pharmaceutical composition in suitable solid form containing the inhibitor. Instillation of the inhibitor may entail installing a solid implant composition into suitable body tissues or cavities. The implant may comprise a matrix of bio-compatible and bio-erodible materials in which particles of a solid inhibitor are dispersed, or in which, possibly, globules or isolated cells of a liquid inhibitor are entrapped. Desirably, the matrix will be broken down and completely absorbed by the body. The composition of the matrix is also preferably selected to provide controlled-, sustained-, and/or delayed release of the inhibitor over extended periods of time, even as much as several months.

A substantial number of the dosage forms described herein may be formulated so as to provide controlled-, sustained-, and/or delayed release of the active ingredient from said dosage form. In an especially preferred aspect of the pharmaceutical compositions of the present invention which provide delayed-, sustained-, and/or controlled-release of the anti-inflammatory selective COX-2 inhibitor active ingredient, there is included all such orally administered dosage forms which produce ≧80% inhibition of COX-2 isozyme activity and result in a plasma concentration of said inhibitor of at least 10 μg/mL for at least 4 hrs; preferably for at least 8 hrs; more preferably for at least 12 hrs; more preferably still for at least 16 hrs; even more preferably still for at least 20 hrs; and most preferably for about 24 hrs. Preferably, there is included the above-described dosage forms which produce ≧80% inhibition of COX-2 isozyme activity and result in a plasma concentration of said inhibitor of at least 15 μg/mL for at least 4 hrs, preferably for at least 8 hrs, more preferably for at least 12 hrs, still more preferably for at least 20 hrs, and most preferably for about 24 hrs. More preferably, there is included the above-described dosage forms which produce ≧90% inhibition of COX-2 isozyme activity and result in a plasma concentration of said inhibitor of at least 20 μg/mL for at least 4 hrs, preferably for at least 8 hrs, more preferably for at least 12 hrs, still more preferably for at least 20 hrs, and most preferably for about 24 hrs.

Accordingly, a useful controlled release dosage form of carprofen in accordance with the present invention is one which maintains a carprofen plasma level greater than 2 μg/mL for most of the day after a single oral dose at 2 mg/lb. Preferred oral controlled release dosage forms of carprofen in accordance with the present invention are ones which maintain a plasma carprofen concentration greater than 10 ug/mL for a period of time greater than that for which an immediate release dosage form of carprofen maintains a comparable plasma level, when said immediate release dosage form and controlled release dosage form are administered at the same dose, e.g. 2, 1.8, 1.6, or 1.4 mg/lb. For instance, preferred 2 mg/lb oral controlled release dosage forms of this invention maintain a plasma carprofen concentration greater than 10 μg/mL for greater than 10.5 hrs.

Immediate release carprofen dosage forms containing doses of 1.8, 1.6, and 1.4 mg/lb maintain a plasma carprofen concentration above 10 μg/mL for 9.5 hrs, 8.5 hrs, and 7.5 hrs, respectively. Preferred 1.8 mg/lb oral controlled release carprofen dosage forms maintain a plasma carprofen concentration above 10 μg/mL for greater than 9.5 hrs. Likewise, the threshold durations for 1.6 mg/lb and 1.4 mg/lb doses are 8.5 hrs and 7.5 hrs, respectively. The performance characteristics for preferred oral controlled release carprofen dosage forms at doses higher than 2 mg/lb or less than 1.4 mg/lb can be similarly calculated, assuming linear pharmacokinetics. More preferred oral controlled release carprofen dosage forms are those which maintain a plasma carprofen concentration greater than 10 μg/mL for a period of time greater than or equal to that observed when an immediate release carprofen dosage form is dosed at any higher dose. These controlled release dosage forms thus maintain at least 80% COX-2 inhibition in canine blood for a period of time longer than that achieved by an immediate release dosage form at a higher dose.

Most preferred oral controlled release carprofen dosage forms are those which are able to maintain plasma carprofen levels above approximately 10 μg/mL for a period of time greater than or equal to the time observed for an immediate release 2 mg/lb carprofen dosage form (10.5 hrs), when said oral controlled release carprofen dosage forms are administered at a dose less than 2 mg/lb. The performance of a 2 mg/lb oral immediate release dosage form is taken as the fundamental standard for purposes of this comparison since 2 mg/lb/day is the currently recommended and accepted efficacious oral dose in accordance with the present invention as described herein.

The term “implant” always denotes a solid pharmaceutical composition containing the anti-inflammatory inhibitor, while the term “depot” usually implies a liquid pharmaceutical composition containing the anti-inflammatory inhibitor, which is deposited in any suitable body tissues or cavities to form a reservoir or pool which slowly migrates to surrounding tissues and organs and eventually becomes systemically distributed. However, these distinctions are not always rigidly adhered to in the art, and consequently, it is contemplated that there is included within the scope of the present invention liquid implants and solid depots, and even mixed solid and liquid forms for each. Suppositories may be regarded as a type of implant, since they comprise bases which are solid at room temperature but melt at body temperature, slowly releasing the active ingredient with which they are impregnated into the surrounding tissue of the body, where the active ingredient becomes absorbed and transported to effect systemic administration.

Systemic administration can also be accomplished by inhalation or insufflation of a powder, i.e., particulate composition containing the inhibitor. For example, the inhibitor in powder form may be inhaled into the lungs using conventional devices for aerosolizing particulate formulations. The inhibitor as a particulate formulation may also be administered by insufflation, i.e., blown or otherwise dispersed into suitable body tissues or cavities by simple dusting or using conventional devices for aerosolizing particulate formulations. These particulate compositions may also be formulated to provide delayed-, sustained-, and/or controlled-release of the anti-inflammatory inhibitor in accordance with well understood principles and known materials.

Other means of systemic administration which may utilize the inhibitors of the present invention in either liquid or solid form include transdermal, intranasal, and opthalmic routes. In particular, transdermal patches prepared in accordance with well known drug delivery technology may be prepared and applied to the skin of a dog to be treated, whereafter the active agent by reason of its formulated solubility characteristics migrates across the epidermis and into the dermal layers of the dog's skin where it is taken up as part of the general circulation of the dog, ultimately providing systemic distribution of the active ingredient over a desired, extended period of time. Also included are implants which are placed beneath the epidermal layer of the skin, i.e. between the epidermis and the dermis of the skin of the dog being treated. Such an implant will be formulated in accordance with well known principles and materials commonly used in this delivery technology, and may be prepared in such a way as to provide controlled-, sustained-, and/or delayed-release of the active ingredient into the systemic circulation of the dog. Such subepidermal (subcuticular) implants provide the same facility of installation and delivery efficiency as transdermal patches, but without the limitation of being subject to degradation, damage or accidental removal as a consequence of being exposed on the top layer of the dog's skin.

Pharmaceutical compositions of special types suitable for oral administration to dogs may also be devised. Pharmaceutical compositions suitable for peroral administration, i.e., ingestion by mouth or administration through the mouth, may be solid or liquid. Preferred peroral dosage forms for systemic administration are solids, e.g., palatable oral compositions such as fast dissolving palatable wafers, tablets, capsules, caplets, lozenges, troches, etc., and liquids, e.g., solutions, suspensions, emulsions, elixirs, tinctures, etc. Pharmaceutical compositions of special types suitable for oral administration to dogs may be used, and include, but are not limited to such items as an oral paste to be delivered to the back of the tongue of the dog being treated, a granular form to be delivered through incorporation in the dog's food, and a chewable form wherein the active ingredient is consumed along with the palatable chew, or a chewable form which may deliver the active ingredient by leaching from the body of the chew which is not consumed, during mastication by the dog being treated. As is known in the art, the formulation of such palatable compositions takes into account canine behavior regarding the extent of mastication of the dosage form which will take place, and the resultant level of dosing.

As with the other routes of administration and corresponding dosage forms described herein, dosage forms intended for oral administration are also suitably formulated to provide controlled-, sustained-, and/or delayed release of the active ingredient. Typically, these would include delayed-release oral tablets, capsules and multiparticulates, as well as enteric-coated tablets and capsules which prevent release and absorption of the active ingredient in the stomach of the dog and facilitate enteric delivery distal to the stomach, ie., in the intestines of the dog. Other typical oral dosage forms would include sustained-release oral tablets, capsules, and multiparticulates which provide systemic delivery of the active ingredient in a controlled manner over a prolonged period of time, e.g., a 24-hour period. Where rapid delivery of the active ingredient is required or desirable, a controlled-release oral dosage form may be prepared in the form of a fast-dissolving tablet, which would also preferably include highly soluble salt forms of the active ingredient.

The description herein of the dosage forms which are contemplated to be within the scope of the present invention has, largely as a matter of convenience, classified such forms into those for local and systemic administration, as well as into solid and liquid forms. However, these distinctions are fairly arbitrary and should not be taken as in any way limiting the scope of the present invention with respect to routes of administration and dosage forms. For example, the description herein has already made it evident that some routes of administration, while ostensibly local, may also have systemic action or consequences. The line drawn herein between liquid and solid dosage forms may also be obscured in actual practice. For example, a suitable oral dosage form for use in the present invention includes encapsulated solutions, a mixed solid and liquid formulation. Microemulsion formulations, also within the scope of the present invention, may also be characterized as a mixed solid and liquid dosage form.

The anti-inflammatory inhibitor can be administered locally to a site of inflammation in a dog to be treated. Local vs. systemic administration entails a more focused vs. a more generalized manner of delivering the anti-inflammatory-inhibitor-containing pharmaceutical composition to the dog suffering from pain and inflammation. However, the use of depots and implants as well as delayed-, sustained-, and controlled-release formulations has tended to blur these distinctions. Accordingly, the above-described liquid and solid pharmaceutical compositions containing the anti-inflammatory inhibitor can, for the most part, be used for local administration as well, but with an emphasis on choosing components for said compositions which will tend to promote absorption of the inhibitor into the local tissues at the site of administration, but which will also tend to prevent infiltration and migration of the inhibitor into more outlying and distant tissues, resulting in systemic carryover.

Local administration is focused on suitable tissues and body cavities into which the anti-inflammatory inhibitor may be injected, infused, implanted, deposited, inserted, instilled, or insufflated. Such administration may include, but is not limited to, that which is intraarterial, intraarticular, intrachondrial, intracostal, intracystic, intra- or transdermal, intrafasicular, intraligamentous, intramedulary, intramuscular, intranasal, intraneural, intraocular, i:e. opthalmic administration, intraosteal, intrapelvic, intrapericardial, intraspinal, intrasternal, intrasynovial, intratarsal, intrathecal, or intravenous.

Pharmaceutical compositions in liquid form containing the inhibitor offer the advantage of permitting injections of the liquid into or in close proximity to the site of inflammation. This is particularly the case where inflamed joints and degenerative joint disease are involved. By injection of the inhibitor directly into the joint, it is possible to achieve a high concentration of inhibitor in a short period of time, thus not only substantially enhancing access of the inhibitor to the site of inflammation, and thus the therapeutic activity of the inhibitor, but also at the same time minimizing the occurrence of untoward adverse reactions that might otherwise occur. The result is a high local concentration of the inhibitor with a correspondingly low systemic carryover concentration.

Injections may also be made of pharmaceutical compositions containing the inhibitor, where the pharmaceutical composition is in delayed-release, controlled-release, or sustained-release-form. These formulations of recognized composition may-be a solids, semi-solids, gels or other liquid/solid combinations in which an erodible matrix or series of coatings is used to provide a continuous release of the inhibitor at a predetermined rate or at variable rates if desired. The terms “extended-release” and “long-acting” as well as others are used to describe these formulations. All of these employ various combinations of bioerodible polymers, e.g., various cellulosic polymers, and natural materials, e.g., corn starch and magnesium stearate, to obtain slow and/or uniform dispensing of the inhibitor contained within the matrix. These pharmaceutical compositions may be injected into the site if suitably liquid or suspendable, or may be delivered by other means if more solid in nature.

The therapeutically effective amount for treating pain and inflammation of inhibitory compounds of Formula (I) is administered to a dog being treated in an amount expressed as milligrams per kilogram of body weight of said dog, per day: “mg/kg/day”. The expression “per day” as used herein should not be interpreted as necessarily requiring that any particular dosage form be administered on a daily basis to the dog being treated. The expression “per day” is merely an indication of the smallest convenient but arbitrary segment of time which is being used as part of the overall unit for measuring the dose of anti-inflammatory inhibitor being administered. The dose, i.e., the therapeutically effective amount for treating pain and inflammation of the inhibitor will usually range from about 0.01 mg/kg/day to about 20.0 mg/kg/day, preferably from about 0.1 mg/kg/day to about 12.0 mg/kg/day, more preferably from about 0.5 mg/kg/day to about 10.0 mg/kg/day, and most preferably from about 0.5 mg/kg/day to about 8.0 mg/kg/day. For instance, a 50 lb. dog weighs 23 kg (1 kg=2.2 lb.), and thus would be treated most preferably with from about 10 mg to about 180 mg of therapeutic agent per day. The fractional amounts are not significant and the dosages would appropriately be rounded to a number which corresponds to unit dosage amounts which are conveniently available. Where the dosage form is, e.g., an injectable liquid, the preferred dosage amounts may be achieved more precisely. On the other hand, where the dosage form is, e.g., an oral tablet, it will be necessary to make more of an approximation of the preferred dosage. Thus, the 10 mg dose could be approximated by halving a 25 mg tablet, and the 180 mg dose could be approximated by using a 100 mg tablet together with a 75 mg tablet or three 25 mg tablets, since these are typical dosage amounts for oral tablets. As will be apparent to those skilled in this art, where the dosage form most frequently employed is the oral tablet and a large number of dogs are treated on a daily basis, added convenience will be obtained through the use of a dispenser containing all of the available dosage amounts of said tablets, e.g., 25 mg, 75 mg, and 100 mg tablets. In this way virtually any preferred dosage amount may be approximated using a combination of said tablets and/or halves thereof.

It is necessary for the skilled artisan, such as a veterinarian, not only to determine the preferred route of administration and the corresponding dosage form and amount, but said artisan must also determine the dosing regimen, i.e., the frequency of dosing. In general terms it is most likely that the choice will be between once-a-day (s.i.d.) dosing and twice-a-day (b.i.d.) dosing, and that the former will provide more rapid and profound therapy, while the latter will provide less profound but more sustained therapy. However, this generalization does not take into account such important variables as the specific type of pain or inflammation involved, the specific therapeutic agent involved and its pharmacokinetics, and the specific patient (dog) involved. For an approved product in the marketplace, much of this information is already provided by the results of clinical studies carried out to obtain such approval. In other cases, such information may be obtained in a straightforward manner in accordance with the teachings and guidelines contained in the instant specification taken in light of the knowledge and skill of the artisan. The results which are obtained can also be correlated with data from corresponding evaluations of an approved product in the same assays.

The above-recited ranges of dosage amounts, which are also recited elsewhere herein, are for racemic mixtures of compounds of Formula (I) having a chiral carbon, or for single compounds of Formula (I) where no chiral carbon atom is present. As will be appreciated by the person of ordinary skill in this art, i.e., a practicing veterinarian or a person with an advanced degree and experience in animal health issues, where other than a racemic mixture of compounds of Formula (I) is involved, the anti-inflammatory therapeutically effective amount will vary. For example, if 85% of the mixture is (S)-enantiomer, that will ordinarily tend to reduce the necessary dosage. These considerations are based on an assumed equal potency, and the fact that the (S)-enantiomer is significantly more active than the (R)-enantiomer. However, the degree of difference between the activities of the two enantiomers must also take into account other differences, especially differences in pharmacokinetics between the two enantiomers, in determining the proper dosage. For example, it has been found that there is a significant difference in clearance rates between the (+)(S) and (−)(R) enantiomers. This, in turn, will have a calculable impact on the amount of active compound to be administered. Ordinarily, such determinations will be made on a case-by-case basis by the artisan, but these are well within the ordinary skill of the art, as is instituting the methods whereby data necessary for the supporting calculations may be obtained.

Typical dosage forms and amounts would include (1) intravenous administration of carprofen at a dose rate of 4.0 mg/kg/day of bodyweight, injected into the right cephalic vein; (2) oral administration of carprofen at a dose rate of 4.0 mg/kg/day of bodyweight as an oral paste syringed on the back of the tongue, given one-hour before feeding; and (3) oral administration of carprofen at a dose rate of 4.0 mg/kg/day of bodyweight as 25 mg, 75 mg, and 100 mg tablet preparations, placed on the back of the tongue of the dog being treated, given one hour before feeding.

The active ingredients of the present invention may also be combined with other therapeutically active ingredients which would be readily apparent to the skilled artisan in this field, and which will usually be determined by the circumstances under which the therapeutic agent of the present invention is administered. For example, where a joint has become seriously inflammed and infected at the same time by microorganisms, e.g., bacteria, fungi, protozoa, virus and the like, the active ingredient of the present invention will desirably be administered in combination with one or more antibiotic, antifungal, antiprotozoal, antiviral or similar therapeutic agents. The active ingredient of the present invention may be administered not only in combination with other NSAIDs of the type described in further detail herein, but in combination as well with inhibitors of other mediators of inflammation, in order to obtain a multi-fold inhibitory action against inflammation. Additional classes of such inhibitors and examples thereof include, e.g., H₁-receptor antagonists; kinin-B₁- and B₂-receptor antagonists; prostaglandin inhibitors such as PGD-, PGF-PGl₂-, and PGE-receptor antagonists; thromboxane A₂ (TXA2-) inhibitors; 5- and 12-lipoxygenase inhibitors; leukotriene LTC₄—, LTD₄/LTE₄—, and LTB₄-inhibitors; PAF-receptor antagonists; gold in the form of an aurothio group together with various hydrophilic groups; immunosuppressive agents, e.g., cyclosporine, azathioprine, and methotrexate; anti-inflammatory glucocorticoids; penicillamine; hydroxychloroquine; anti-gout agents, e.g., colchicine, xanthine oxidase inhibitors, e.g., allopurinol, and uricosuric agents, e.g., probenecid, sulfinpyrazone, and benzbromarone.

Because inflammation is most prevalent among geriatric dogs, it will be appreciated by those skilled in the art that the anti-inflammatory agents of the present invention may also be administered in combination with therapeutic agents intended for the treatment of disease conditions, syndromes and symptoms which are also found in abundance in older dogs. Such therapeutic agents and the conditions which they are used to treat include, e.g., cognitive therapeutics to counteract memory loss and impairment. Another large class of such therapeutic agents includes; anti-hypertensives and other cardiovascular drugs intended to offset hypertension, myocardial ischemia including angina, congestive heart failure, and myocardial infarction, e.g., diuretics, vasodilators such as hydralazine, β-adrenergic receptor antagonists such as propranolol, angiotensin-II converting enzyme inhibitors (ACE-inhibitors) such as enalapril used to treat geriatric dogs with mitral insufficiency, and enalapril alone and in combination with neutral endopeptidase inhibitors, angiotensin II receptor antagonists such as losartan, renin inhibitors, calcium channel blockers such as nifedipine, sympatholytic agents such as methyldopa, Q₂-adrenergic agonist such as clonidine, α-adrenergic receptor antagonists such as prazosin, and HMG-CoA-reductase inhibitors (anti-hypercholesterolemics) such as lovastatin.

Still other classes of such therapeutic agents include antineoplastic agents, especially antimitotic drugs including the vinca alkaloids such as vinblastine and vincristine, for treating various cancers; therapeutic agents for treating renal failure; anti-obesity drugs for treating excess weight problems in dogs; anti-parasitic drugs for treating both endo- and ecto-parasites which commonly afflict dogs; and anti-pruritic drugs for treating various types of pruritis in dogs.

Other types of drugs which can be used in combination with the anti-inflammatory agents of the present invention include growth hormone secretagogues; strong analgesics; local and systemic anesthetics; and H₂-receptor antagonists and other gastroprotective agents. It will be recognized by those of ordinary skill in this art that some of the above combinations of therapeutic agents will be used most frequently to treat various acute conditions in dogs, e.g., bacterial infections occurring simultaneously with degenerative joint disease. However, there would be an equal if not greater intererst on the part of such skilled persons in treating chronic conditions in dogs.

In accordance with a regimen which would be used for this purpose, it is contemplated that the anti-inflammatory agents of the present invention would be administered in combination with other medications used on a regularly scheduled basis for treating chronic conditions such as osteoarthritis. It is also envisioned that adminstration in combinations could assume a number of different forms and still be within the scope of the present invention. For example, the anti-inflammatory agents of the present invention might simply be formulated with one or more of the other therapeutic agents which are to form the intended combination, into a convenient dosage form, such as an oral tablet, containing all of the drugs forming the combination. Varying half-lives for the different drugs could be accommodated by the person skilled in preparing formulations by creating controlled-release forms of said drugs with different release times so that relatively uniform dosing was achieved. A medicated feed used as the dosage form could also be prepared in accordance with well known principles in the art of formulation, in which the drugs used in the combination were simply present together in admixture in the feed composition. The present invention also contemplates co-administration in which the combination of drugs is achieved by the simultaneous administration of the drugs to be given in combination. Such co-administration could even be by means of different dosage forms and routes of administration. The present invention further contemplates the use of such combinations in accordance with different but regular and continuous dosing schedules whereby desired plasma levels of the drugs involved were maintained in the dog being treated, even though the individual drugs making up the combination were not being administered to said dog simultaneously. All such combinations would be well within the skill of the art to devise and administer.

The methods and compositions of the present invention are useful for treating or preventing pain and/or inflammation in dogs. Pain, which is a more or less localized sensation of discomfort, distress, or agony, resulting from the stimulation of specialized nerve endings, may occur as or be viewed as separate and distinct from inflammation. For example, chronic pain and pain induced and/or associated with surgery, e.g., peri-operative and postoperative pain, initially may have little association with inflammation. Opioid analgesics are effective in the treatment of postoperative pain but do not affect inflammation. However, NSAIDs can be superior to such opioid analgesics in the treatment of some forms of postoperative pain, and they are particularly effective in settings in which inflammation has caused sensitization of pain receptors to normally painless mechanical or chemical stimuli. While NSAIDs inhibit the biosynthesis and release of protaglandins, which are inflammatory mediators, there is data to suggest that the analgesic affect of NSAIDs may occur by way of mechanisms other than inhibition of protaglandin synthesis.

Accordingly, the present invention has been described in terms of being useful in the treatment and prevention of pain and inflammation, since these most often occur together in tissue injury and disease processes and conditions mediated by COX-2. However, there is no intention to inextricably link pain and inflammation with regard to the usefulness of the present invention in treating and preventing them; and they are thus to be regarded in the context of the instant specification as being the object, either individually and separately, or together in combination, of the methods, compositions and other facets of the present invention.

The inflammatory process itself may have a number of precipitating causes, including infectious agents, ischemia, antigen-antibody interactions, and thermal or other physical injury. The response to each of these causes is characteristically different, but they all have a strong commonality. Clinical symptoms include erythema, edema, tenderness and pain. Three distinct phases can be recognized, but each of these is mediated by different mechanisms. The first, acute transient phase involves local vasodilation and increased capillary permeability; the second, delayed, subacute phase involves infiltration of leukocytes and phagocytic cells; and the third, chronic proliferative phase involves tissue degeneration and fibrosis. NSAIDs as a therapeutic class of anti-inflammatory agents, appear to act by inhibiting the enzymatic production and release of prostaglandins, which participate in the pathogenesis of inflammation and fever. However, the NSAIDs do not inhibit the formation of eicosanoids such as the leukotrienes, which also contribute to inflammation, nor do they interfere with the formation of numerous other mediators of inflammation.

It has been discovered, in accordance with the present invention, that the carprofen genus of compounds of Formula (I), and especially carprofen itself, and more especially the (S)-enantiomer of carprofen, alone among the NSAIDs having a carboxylic acid moiety, have a surprising and unexpectedly high degree of selectivity for the COX-2 isozyme. While this particular isozyme is an important mediator of inflammation, there are many other important mediators of inflammation that either have no interaction with NSAIDs, or no well understood relationship to the action of NSAIDs. Such mediators include several classes of leukocytes; cell adhesion molecules; soluble mediators such as C5a, PAF and leukotriene B₄; cytokines such as IL-1 and TNF; growth factors such as GM-CSF and TGF-β₁; histamine, bradykinin and 5-HT. While the compounds of Formula (I) are shown herein to be unique inhibitors of COX-2, there is no intention thereby to be bound to any particular mechanism of action by which the compounds of Formula (I) might exert their anti-inflammatory activity.

Indeed, it has been pointed out further above that the anti-inflammatory mode of action of carprofen and the other compounds of Formula (I) is not well understood, and that there has been speculation heretofore that the actual mode of action might involve neutrophils, also known as polymorphonuclear leukocytes. PAF stimulates such cells to aggregate, to release leukotrienes and lysosomal enzymes, and to generate superoxide, all of which promote inflammation.

The uniqueness of carprofen and the compounds of Formula (I) among NSAIDs has already been referred to in general terms. While the compounds of Formula (I) are clearly NSAIDs, they are not readily placed in any of the recognized chemical classification of NSAIDs:

| | |
|---|---|
| Salicylic acids | aspirin; |
| p-Aminophenols | acetaminophen; |
| Indole/indene acetic acids | Indomethacin, sulindac, etodolac; |
| Heteroaryl acetic acids | tolmetin, diclofenac, ketorolac; |
| Arylpropionic acids | ibuprofen, naproxen, flurbiprofen, |
| | ketoprofen; |
| Anthranilic acids | mefenamic acid, meclofenamic acid; |
| Enolic acids | oxicams, e.g., piroxicam, tenoxicam; |
| Pyrazolidinediones | phenylbutazone; |
| Alkanones | nabumetone. |

Carprofen and the compounds of Formula (I), although they are propionic acids, do not belong to the subclass of arylpropionic acids because the carbazole group of the carprofens is heteroaryl, not aryl. The carprofens do not belong to the subclass of heteroaryl acetic acids, because the carprofens are propionic acids, not acetic acids. The carprofens cannot be placed in any of the other subclasses without doing some violence to the bases of classification. The only NSAID approved for treatment of humans which is recognized to have human COX-2 selective activity is nabumetone in the above list, which is not an acid at all but a 2-butanone. Although the active species is the acid metabolite, this metabolite has onty a small fraction of the COX-2 selectivity of carprofen in dogs.

Exemplary of the numerous classical NSAIDs within this broad class are the compounds set out in the below table, which gives the common and IUPAC names of each compound and its structure. All of the enumerated compounds appear in USP Dictionary of USAN and International Drug Names, 1995, C. A. Fleeger, ed., United States Pharmacopeial Convention, Inc., Rockville, Md. The USAN (United States Adopted Names) program produces simple and useful non-proprietary names for drugs, and the name-selection process is initiated when the drug enters the clinical investigation stage. The name and structure of carprofen are given at the beginning of the table in order to facilitate comparison.

TABLE 1

| COMMON NAME | IUPAC NAME | STRUCTURE |
|---|---|---|
| Carprofen | 6-chloro-α-methyl-9H-carbazole-2-acetic acid | 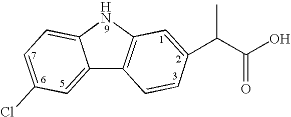 |
| Alclofenac | 3-chloro-4-(2-propenyloxy)-benzene acetic acid | 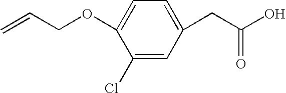 |
| Aspirin | 2-(acetyloxy)-benzoic acid | 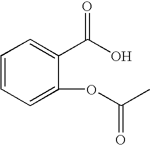 |
| Diflunisal | 2′,4′-difluoro-4-hydroxy-3- biphenylcarboxylic acid | 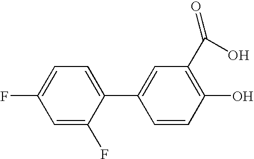 |
| Diclofenac | 2-[(2,6-dichlorophenyl)amino]- benzeneacetic acid | 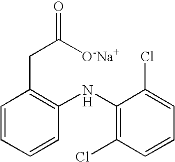 |
| Etodolac | 1,8-diethyl-1,3,4,9-tetrahydro-pyrano[3,4- b]indole-1-acetic acid | 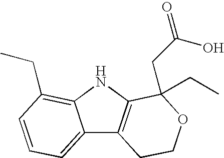 |
| Felbinac | [1,1′-biphenyl]-4-acetic acid | 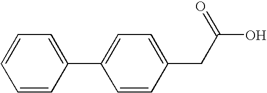 |
| Flufenamic acid | 2-{[3-(trifluoromethyl)phenyl]amino}- benzoic acid | 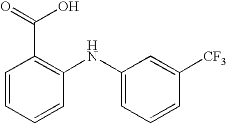 |
| Flunixin Meglumine (Banamine ®)¹ | 2-{[2-methyl-3-(trifluoromethyl)- phenyl]amino}-3-pyridinecarboxylic acid; compounded with 1-deoxy-1- (methylamino)-D-glucitol(1:1) | 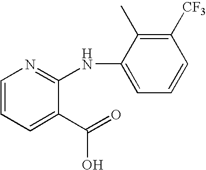 |
| Flurbiprofen | 2-fluoro-α-methyl-[1,1′-biphenyl]-4-acetic acid | 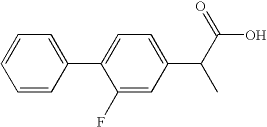 |
| Ibuprofen | α-methyl-4-(2-methylpropyl) benzeneacetic acid | 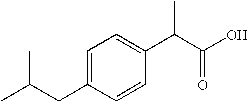 |
| Ilonidap | 6-chloro-5-fluoro-2,3-dihydro-(hydroxy-2- thienylmethylene)-2-oxo-1H-indole-1- carboxamide | 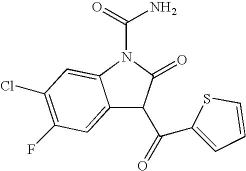 |
| Indomethacin | 1-(4-chlorobenzoyl)-5-methoxy-2-methyl- 1H-indole-3-acetic acid | 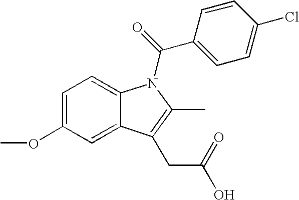 |
| Indoprofen | 4-(1,3-dihydro-1-oxo-2H-isoindol-2-yl)- benzeneacetic acid | 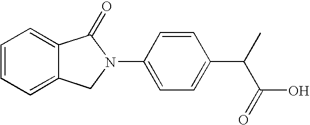 |
| Isoxepac | 6,11-dihydro-11-oxo-dibenz[b,e]oxepin- 2-acetic acid | 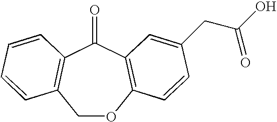 |
| Ketoprofen (Ketofen ®)¹ | 3-benzoyl-α-methyl-benzeneacetic acid | 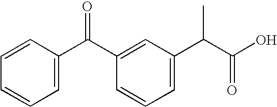 |
| Meclofenamic acid (Arquel ®)¹ | 2-[(2,6-dichloro-3-methylphenyl)amino]- benzoic acid | 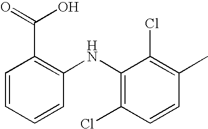 |
| Mefenamic acid | 2-[(2,3-dimethylphenyl)amino]-benzoic acid | 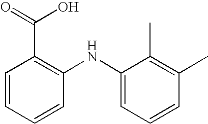 |
| Meloxicam (Metacam ®) | 4-hydroxy-2-methyl-N-(5-methyl-2- thiazolyl)-2H-1,2-benzothiazine-3- carboxamide | 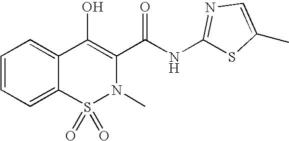 |
| Nabumetone and 6-MNa | 4-(6-methoxy-2-naphthalenyl)-2- butanone; prodrug; metabolized in vivo to active 6-methoxy-2-naphthyl-acetic acid (6-MNA) depicted at right | 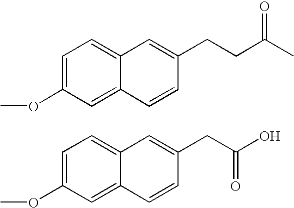 |
| Naproxen | 6-methoxy-α-methyl-2-naphthaleneacetic acid | 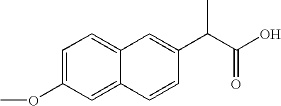 |
| Niflumic acid | 2-[3-(trifluoromethyl)anilino]nicotinic acid | 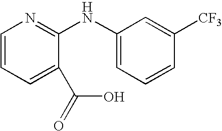 |
| Nimesulide (Sulidene ®)¹ | 4′-nitro-2′-phenoxymethanesulfonanilide | 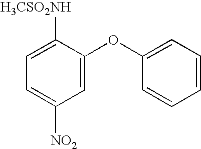 |
| NS-398 | N-[2-(cyclohexyloxy)-4-nitrophenyl]- methanesulfonamide | 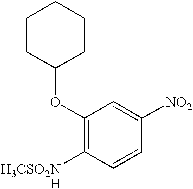 |
| Phenylbutazone | 4-butyl-1,2-diphenyl- 3,5pyrazolidinedione | 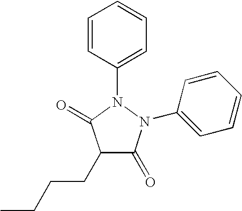 |
| Piroxicam (Feldene ®)¹ | 4-hydroxyy-2-methyl-N-2-pyridinyl-2H- 1,2-benzothiazine-3-carboxamide-1,1- dioxide | 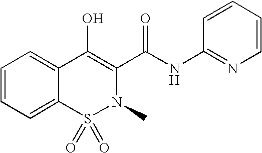 |
| Tenidap | 5-chloro-2,3-dihydro-3-(hydroxy-2- thienylmethylene)-2-oxo-1H-indole-1- carboxamide | 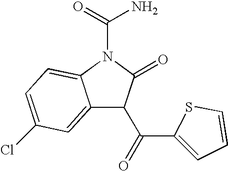 |
| Tolfenamic acid | N-(3-chloro-o-tolyl)-anthranilic acid | 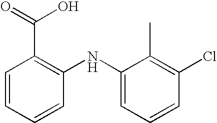 |
| Vedaprofen | (±)-4-cyclohexy-α-methyl-1-naphthalene acetic acid | 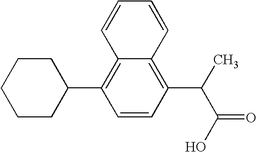 |

¹Registered Trademark; approved for use in dogs outside the United States.

### DESCRIPTION OF PREFERRED EMBODIMENTS

The carprofen genus of compounds, characterized by an α-methyl-acetic acid functionality, has many times greater COX-2 selectivity in dogs than any of the carboxyl-containing or sulfonyl- or sulfonamide-containing NSAIDs set out in the above table. In order to demonstrate this unexpected property, a comparison was made between the COX-2 selectivity of carprofen, a compound of the present invention, and the COX-2 selectivity of certain selected compounds from the above table. The results are illustrated in the below-described working examples.

As described above, the selectivity for COX-2 is conventionally determined as the ratio of COX-1 inhibition to COX-2 inhibition, or vice versa. In the present description, the ratio of COX-1 inhibition to COX-2 inhibition in dogs is utilized essentially for the sake of simplicity. Both inhibition values are IC₅₀ values, which means that the more active a test compound is, the smaller will be the IC₅₀ value. This, in effect, inverts the ratio so that where it is COX-1:COX-2, and a test compound is very selective for canine COX-2, the ratio will be that of a larger number over a much smaller number. Thus, the most canine COX-2 selective test compounds will be those with the highest ratio numbers.

### EXAMPLE 1

Comparative Evaluation of Canine cyclo-oxygenase-1 and -2 Inhibition by Carprofen and Other NSAIDs

Protocol for Evaluation of Canine COX-1 Activity

Test drug compounds were solubilized and diluted the day before the assay was to be conducted with 0.1 mL of DMSO/9.9 mL of Hank's balanced salts solution (HBSS), and stored overnight at 4° C. On the day that the assay was carried out, citrated blood was drawn from a donor dog, centrifuged at 190×g for 25 min at room temperature, and the resulting platelet-rich plasma was then transferred to a new tube for further procedures. The platelets were washed by centrifuging at 1500×g for 10 min at room temperature. The platelets were washed with platelet buffer comprising Hank's buffer (Ca free) with 0.2% bovine serum albumin (BSA) and 20 mM HEPES. The platelet samples were then adjusted to 1.5×10 mL, after which 50 μl of calcium ionophore (A23187) together with a calcium chloride solution were added to 50 μl of test drug compound dilution in plates to produce final concentrations of 1.7 μM A23187 and 1.26 mM Ca. Then, 100 μl of canine washed platelets were added and the samples were incubated at 37° C. for 15 min, after which the reaction was stopped by adding 20 μl of 77 mM EDTA. The plates were then centrifuged at 2000×g for 10 min at 40° C., after which 50 μl of supernatant was assayed for thromboxane B₂ (TXB₂) by enzyme-immunoassay (EIA). The pg/mL of TXB₂ was calculated from the standard line included on each plate, from which it was possible to calculate the percent inhibition of COX-1 and the IC₅₀ values for the test drug compounds.

Protocol for Evaluation of Canine COX-2 Activity

A canine histocytoma (macrophage-like) cell line from the American Type Culture Collection designated as DH82, was used in setting up the protocol for evaluating the COX-2 inhibition activity of various test drug compounds. There was added to flasks of these cells 10 μg/mL of LPS, after which the flask cultures were incubated overnight. The same test drug compound dilutions as described above for the COX-1 protocol were used for the COX-2 assay and were prepared the day before the assay was carried out. The cells were harvested from the culture flasks by scraping, and were then washed with minimal Eagle's media (MEM) combined with 1% fetal bovine serum, centrifuged at 1500 rpm for 2 min, and adjusted to a concentration of 3.2×10⁵ cells/mL. To 50 μl of test drug dilution there was added 50 μl of arachidonic acid in MEM to give a 10 μM final concentration, and there was added as well 100 μl of cell suspension to give a final concentration of 1.6×1 cells/mL. The test sample suspensions were incubated for 1 hr and then centrifuged at 1000 rpm for 10 min at 4° C., after which 50 μl aliquots of each test drug sample were delivered to EIA plates. The EIA was performed for prostaglandin E₂ (PGE₂), and the pg/mL concentration of PGE₂ was calculated from the standard line included on each plate. From this data it was possible to calculate the percent inhibition of COX-2 and the IC₅₀ values for the test drug compounds. Repeated investigations of COX-1 and COX-2 inhibition were conducted over the course of several months. The results were averaged, and a single COX-1:COX-2 ratio was calculated. The data obtained, together with an indication of the number of tests conducted for each test sample, are set forth in the following table of values.

TABLE 2

| | No.of | COX-1 | COX-2 | COX-1/COX-2 |
|---|---|---|---|---|
| NSAID | tests | IC₅₀ μM | IC₅₀ μM | Ratio |
| Carprofen (rac.) | 9 | 13.2 | 0.102 | 129 |
| Carprofen (S-) | 3 | 6.71 | 0.0371 | 181 |
| Carprofen (R-) | 4 | >25.0 | 5.97 | >4.19 |
| Flufenamic acid | 6 | 2.31 | 0.0475 | 48.6 |
| Nimesulide | 6 | 2.15 | 0.0565 | 38.0 |
| Niflumic acid | 6 | 1.03 | 0.0464 | 22.2 |
| Meclofenamic acid | 5 | 0.737 | 0.0478 | 15.4 |
| Tolfenamic acid | 4 | 0.206 | 0.0137 | 15.0 |
| Naproxen | 3 | 7.08 | 0.626 | 11.3 |
| Mefenamic acid | 4 | 0.403 | 0.0362 | 11.1 |
| Felbinac | 3 | 2.54 | 0.362 | 7.01 |
| 6-MNA | 6 | 28.3 | 4.21 | 6.72 |
| NS-398 | 7 | 0.587 | 0.137 | 4.28 |
| Flurbiprofen | 4 | 0.505 | 0.123 | 4.10 |
| Diclofenac | 3 | 0.246 | 0.0778 | 3.16 |
| Meloxicam | 5 | 0.891 | 0.307 | 2.90 |
| Phenylbutazone | 5 | >10.0 | 3.79 | >2.46 |
| Ibuprofen | 4 | 1.03 | 0.391 | 2.63 |
| Tenidap | 12 | 0.469 | 0.228 | 2.06 |
| Alclofenac | 3 | 13.2 | 7.41 | 1.78 |
| Ilonidap | 16 | 0.472 | 0.270 | 1.75 |
| Flunixin | 5 | 0.00768 | 0.0121 | 0.635 |
| Etodolac | 3 | 1.33 | 2.57 | 0.517 |
| Piroxicam | 6 | 0.223 | 0.585 | 0.381 |
| Ketoprofen | 5 | 0.0286 | 0.123 | 0.232 |
| Indomethacin | 6 | 0.0558 | 0.366 | 0.152 |
| Aspirin | 3 | 34.3 | >100 | <0.343 |
| Vedaprofen | | | | |

### EXAMPLE 2

Canine Whole Blood ex vivo Determinations of COX-1 and COX-2 Activity Inhibition by Carprofen

The objective of this study was to evaluate the inhibitory potency of carprofen against COX-1 and COX-2 activity using an ex vivo procedure on canine whole blood. Three dogs were dosed with 10 mg/kg of racemic 6-chloro-α-methyl-carbazole-2-acetic acid (carprofen) administered by mouth (PO) in capsule dosage form, three dogs were dosed with 2 mg/kg of carprofen on the same basis, and three dogs were untreated. A zero-hour blood sample was collected from all dogs in the study prior to dosing, followed by 1-, 3-, and 6-hour post-dose blood sample collections. Test tubes were prepared containing 2 μL of either (A) calcium ionophore A23187 giving a 50 μM final concentration, which stimulates the production of thromboxane B₂ (TXB₂) for COX-1 activity determination; or of (B) lipopolysaccharide (LPS) to give a 10 μg/mL final concentration, which stimulates the production of prostaglandin E₂ (PGE₂) for COX-2 activity determination. Test tubes used as controls contained vehicle and were unstimulated by the addition of any agent. A 500 μL sample of blood was added to each of the above-described test tubes, after which they were incubated at 37° C. for one hr in the case of the calcium ionophore-containing test tubes, and overnight in the case of the LPS-containing test tubes. After incubation, 10 μL of EDTA was added to give a final concentration of 0.3%, in order to prevent coagulation of the plasma which sometimes occurs after thawing frozen plasma samples. The incubated samples were centrifuged at 4° C. and the resulting plasma sample of ˜200 μL was collected and stored at −20° C. in polypropylene 96-well plates. In order to determine endpoints for this study, enzyme immunoassay (EIA) kits available from Cayman were used to measure production of TXB₂ and PGE₂, utilizing the principle of competitive binding of tracer to antibody and endpoint determination by colorimetry. Plasma samples were diluted to approximate the range of standard amounts which would be supplied in a diagnostic or research tools kit, i.e., 1/500 for TXB₂ and 1/750 for PGE₂

The data set out in Table 3 further below demonstrate the percent inhibition of COX-1 and COX-2 activity based on their zero hour values. The data is expressed as treatment group averages in pg/mL of TXB₂ and PGE₂ produced per sample. Plasma dilution was not factored in said data values.

The data in Table 3 show that at the 2 mg/kg dose there was significant COX-2 inhibition at all timepoints. At 3- and 6-hours post-dose, there is observed to have been a slight decline in COX-2 inhibition compared to the data obtained for the 10 mg/kg dose. The data in Table 3 also show that at the 2 mg/kg dose there was no significant inhibition of COX-1 activity at any of the timepoints involved. This result was consistent with the observed excellent toleration of carprofen by the dogs in the study. The data for the 10 mg/kg dose show that there was complete inhibition of COX-2 activity at every timepoint, and very strong inhibition of COX-1 activity beginning at 1 hr and plateauing over the 3- to 6-hour timepoints. Accordingly, the data in Table 3 clearly demonstrate that at a 2 mg/kg dosage concentration carprofen possesses good COX-2 selectivity. As the dose is increased from 2 to 10 mg/kg, increasing inhibition of COX-1 activity becomes evident.

TABLE 3
COX-1 ACTIVITY INHIBITION - Group Averages

| | TXB₂ Pg/mL/Well | | | | Percent Inhibition | | |
|---|---|---|---|---|---|---|---|
| | 0- | 1- | 3- | 6- | 1- | 3- | 6- |
| Hour | hour | hour | hour | hour | hour | hour | hour |
| Untreated | 102 | 134 | 77 | 65 | 0% | 25% | 36% |
| 2 mg/kg | 72 | 75 | 66 | 46 | 0% | 8% | 36% |
| 10 mg/kg | 51 | 31 | 6 | 7 | 39% | 88% | 86% |

COX-2 ACTIVITY INHIBITION - Group Averages

| | PGE₂ Pg/mL/Well | | | | Percent Inhibition | | |
|---|---|---|---|---|---|---|---|
| | 0- | 1- | 3- | 6- | 1- | 3- | 6- |
| Hour | hour | hour | hour | hour | hour | hour | hour |
| Untreated | 393 | 900 | 349 | 405 | 0% | 11% | 0% |
| 2 mg/kg | 511 | 33 | 98 | 190 | 94% | 81% | 63% |
| 10 mg/kg | 336 | 19 | 12 | 12 | 94% | 96% | 96% |

COX inhibition is observed when the measured percent inhibition is greater than that measured for untreated controls. The percent inhibition in the above table is calculated in a straightforward manner in accordance with the following equation:

$$\% I nhibition ( 1 \text{-} hour ) = \frac{( {PGE}_{2} at t = 0 ) - ( {PGE}_{2} at t = 1 )}{( {PGE}_{2} at t = 0 )} \times 100$$

### EXAMPLE 3

Canine Whole Blood Ex Vivo Determinations of COX-2 Activity Inhibition by Carprofen

This study followed the procedures described in Example 2 above, but with some modifications of detail below described.

Three dogs were dosed with 2 mg/kg of racemic 6-chloro-α-methyl-carbazole-2-acetic acid (carprofen) administered by mouth (PO) in tablet dosage form at zero hour; three dogs were dosed with 4 mg/kg of carprofen on the same basis at zero hour; and three dogs were untreated. A zero-hour blood sample was collected from all dogs in the study prior to dosing, followed by 2- and 4-hour post-dose blood sample collections. Test tubes were prepared containing 2 μL of lipopolysaccharide (LPS) to give a 10 μg/mL final concentration. Untreated test tubes were used as controls. A 500 μL sample of blood was added to each of the above-described test tubes, after which they were incubated at 37° C. overnight. After incubation 10 mL of EDTA was added to give a final concentration of 0.3%. The incubated samples were centrifuged at 4° C. and the resulting plasma sample of 200 μL was collected and stored at −20° C. in propylene 96-well plates. Enzyme immunoassay (EIA) kits available from Cayman were used to measure production of PGE₂, with endpoint determination by colorimetry. Plasma samples were diluted to 1/750 for PGE₂.

The plasma samples were also assayed for total plasma carprofen concentration using HPLC with a 5 micron, 100×4.6 mm Chromtech chiral AGP column and a mobile phase composed of 10:90 v/v 2-propanol:0.1M phosphate buffer pH 6.0, and fluorescence detection (285 nm excitation, 345 nm emission). Plasma samples (0.2 mL) were buffered with 0.05 M citric acid pH 5.1 following the addition of (S)-naproxen as the internal standard, then were extracted with 4:1 v/v diethyl ether:dichloromethane. The ether layer was separated, then back-extracted with 0.005 M Na₂CO₃, after which the organic phase was discarded by aspiration. The aqueous phase was buffered with 0.05 M citric acid pH 5.1, then was again extracted with diethyl ether:dichloromethane. The ether was then transferred to clean tubes, evaporated under a stream of nitrogen, and the residue reconstituted in HPLC mobile phase for analysis.

The data set out in Table 4 further below show production of PGE₂ (pg/mL/well), percent inhibition of COX-2 activity based on each dog's zero hour values, as well as plasma exposure for individual dogs.

The data in Table 4 show that at 2- and 4-hours post-dose, there is observed to have been a small difference between the doses, with the 4 mg/kg dose showing slightly greater COX-2 inhibition. Ex vivo COX-2 inhibition is well correlated with plasma concentrations of carprofen.

TABLE 4
COX-2 ACTIVITY INHIBITION and CARPROFEN
PLASMA CONCENTRATIONS

| | | | | | | | Plasma | |
|---|---|---|---|---|---|---|---|---|
| | | PGE₂ | | | Percent | | concentration | |
| | | Pg/mL/Well | | | Inhibition | | ug/mL | |
| Dog | Dose | 0- | 2- | 4- | 2- | 4- | 2- | 4- |
| number | (mg/kg) | hour | hour | hour | hour | hour | hour | hour |
| 35769 | 0 | 426 | 340 | 439 | 20% | 0% | — | — |
| 05690 | 0 | 130 | 239 | 219 | 0% | 0% | — | — |
| 06044 | 0 | 860 | 660 | 632 | 23% | 27% | — | — |
| 35773 | 2 | 293 | 35 | 69 | 88% | 77% | 21.9 | 13.4 |
| 35742 | 2 | 541 | 113 | 223 | 79% | 59% | 8.6 | 5.7 |
| 36184 | 2 | 409 | 46 | 90 | 89% | 78% | 17.2 | 10.6 |
| 05717 | 4 | 471 | 25 | 20 | 95% | 96% | 37.6 | 29.1 |
| 05704 | 4 | 393 | 21 | 25 | 96% | 94% | 29.9 | 21.1 |
| 36185 | 4 | 622 | 14 | 14 | 98% | 98% | 29.0 | 27.0 |

The percent inhibition in the above table is calculated in a straightforward manner in accordance with the following equation:

$$\% I nhibition ( 2 \text{-} hour ) = \frac{( {PGE}_{2} at t = 0 ) - ( {PGE}_{2} at t = 2 )}{( {PGE}_{2} at t = 0 )} \times 100$$

### EXAMPLE 4

Canine In Vivo Determinations of COX-2 Activity Inhibition by Carprofen in a Carrageenan-Induced Inflammation Model

This objective of this study was to monitor in vivo COX-2 activity in a subcutaneously implanted chamber during an induced inflammation. The COX-2 enzyme can be detected by western analysis as early as 5 hours after carrageenan treatments (T. Kirchner et al., J. Pharmacol. Exp. Ther. (1997) 282, 1094-1101). In addition, COX-1 activity may be simultaneously determined by ex vivo methods as described in Example 2. Six beagle dogs had polyethylene “whiffle” golf balls approximately 4.2 cm in outer diameter surgically implanted under the skin just behind the shoulder blades. They were allowed to recover from the surgery for 1 month before being assigned to experimental groups.

The experiment was run in two replicates. On the day of the experiment, three dogs were given 2 mg/kg of carprofen PO 30 min. before the start of the inflammation and the other three dogs were used as controls. The hair around the ball was shaved and the area was probed to locate a hole in the whiffle ball. This area was then marked and it was sterilized with 2% iodine tincture. A needle was inserted into the hole and 1.5 cc of fluid was removed, hereinafter referred to as the exudate fluid “EF.” After removing 1.5 cc of EF, 1.5 cc of a 0.33% carrageenan solution in H₂O was added to the ball to induce an inflammatory event. EF samples were taken at 0, 5, and 24 hours after carrageenan injection. Blood samples were also taken so that an ex vivo COX-1 whole blood assay could be run, as described in Example 2.

The EF sample needed to be purified prior to assay, due to the low amount of PGE₂ found in the in vivo sample. This was accomplished by the use of 4 mL PGE₂ affinity columns (Cayman Chemical). The column was first washed with 10 mL of 0.1 M phosphate buffer then 10 mL water. The EF was then diluted 1:5 with 0.1 M phosphate buffer and added to the column. The column was washed with 10 mL of the phosphate buffer, then 10 mL of water. Finally, the PGE₂ was removed from the column with 2.5 mL 95% ethanol. The sample was evaporated under a stream of nitrogen and then diluted for analysis of PGE₂ by the Cayman EIA PGE₂ (COX-2) kit, as described in Examples 2 and 3.

Table 5 illustrates that carrageenan induced almost a 4-fold increase in PGE₂ synthesis in the whiffle ball over background levels. This increase was seen at 5 hours and remained until at least 24 hours. The carprofen dosed animals at 2 mg/kg show near total inhibition of PGE₂ synthesis that was statistically significant at 5 hours and 24 hours, with p-values of 0.013 and 0.015, respectively. The ex vivo COX-1 data showed no inhibition of TXB2 at any of the timepoints tested. This indicates that carprofen showed in vivo C0QX-2 inhibition at the same time it showed no effect on ex vivo COX-1 activity.

TABLE 5
IN VIVO COX-2 ACTIVITY INHIBITION - Group Averages

| | PGE₂ | | | | |
|---|---|---|---|---|---|
| Dose | Pg/mL/Well | | | Percent Inhibition | |
| (mg/kg) | 0-hour | 5-hour | 24-hour | 5-hour | 24-hour |
| 0 | 3.4 | 17.1 | 13.0 | 0% | 0% |
| 2 | 6.5 | 1.9 | 3.2 | 71% | 51% |

### EXAMPLE 5

Computation of Carprofen Controlled Release Dosage Form Oral Drug Delivery Input Rates and Doses Which Give Cox-2 Inhibition of 80% in Dogs for a Length of Time Longer Than an Immediate Release Carprofen Dosage Form

A study was carried out to obtain data from which to determine useful, preferred, more preferred, and most preferred oral carprofen controlled release dosage forms for use in the present invention.

The plasma samples collected from dogs in above-recited Example 3 were also assayed for total plasma carprofen concentration using HPLC with a 5 micron, 100×4.6 mm Chromtech chiral AGP column and a mobile phase composed of 10:90 v/v 2-propanol:0.1M phosphate buffer pH 6.0, and fluorescence detection (285 nm excitation, 345 nm emission). Plasma samples (0.2 mL) were buffered with 0.05 M citric acid pH 5.1 following the addition of (S)-naproxen as the internal standard, then were extracted with 4:1 v/v diethyl ether:dichloromethane. The ether layer was separated, then back-extracted with 0.005 M Na₂CO₃, after which the organic phase was discarded by aspiration. The aqueous phase was buffered with 0.05 M citric acid pH 5.1, then was again extracted with diethyl ether:dichloromethane. The ether was then transferred to clean tubes, evaporated under a stream of nitrogen, and the residue reconstituted in HPLC mobile phase for analysis.

The carprofen plasma levels were plotted against % COX-2 inhibition (from Example 3). Inspection of this plot indicated that 80% COX-2 inhibition occurred at a plasma carprofen concentration of approximately 10 μg/mL, and that 50% COX-2 inhibition occurred at a plasma carprofen concentration of approximately 2 μg/mL, and that 90% COX-2 inhibition occurred at a plasma carprofen concentration of approximately 20 μg/mL.

Following oral dosing to dogs of an immediate release carprofen formulation at a dose of 2 mg/lb, plasma carprofen concentrations were above 10 μg/mL for approximately 10.5 hr. In accordance with the present invention, 2 mg/lb/day is within the range of preferred doses of carprofen.

A useful controlled release dosage form of carprofen in accordance with the present invention is one which maintains a carprofen plasma level greater than 2 pg/mL for most of the day after a single oral dose at 2 mg/lb.

Preferred oral controlled release dosage forms of carprofen in accordance with the present invention are ones which maintain a plasma carprofen concentration greater than 10 μg/mL for a period of time greater than that for which an immediate release dosage form of carprofen maintains a comparable plasma level, when said immediate release dosage form and controlled release dosage form are administered at the same dose, e.g. 2, 1.8, 1.6, or 1.4 mg/lb. For instance, preferred 2 mg/lb oral controlled release dosage forms of this invention maintain a plasma carprofen concentration greater than 10 μg/mL for greater than 10.5 hrs.

Immediate release carprofen dosage forms containing doses of 1.8, 1.6, and 1.4 mg/lb maintain a plasma carprofen concentration above 10 μg/mL for 9.5 hrs, 8.5 hrs, and 7.5 hrs, respectively.

Preferred 1.8 mg/lb oral controlled release carprofen dosage forms maintain a plasma carprofen concentration above 10 μg/mL for greater than 9.5 hrs. Likewise, the threshold durations for 1.6 mg/lb and 1.4 mg/lb doses are 8.5 hrs and 7.5 hrs, respectively. The performance characteristics for preferred oral controlled release carprofen dosage forms at doses higher than 2 mg/lb or less than 1.4 mg/lb can be similarly calculated, assuming linear pharmacokinetics.

More preferred oral controlled release carprofen dosage forms are those which maintain a plasma carprofen concentration greater than 10 μg/mL for a period of time greater than or equal to that observed when an immediate release carprofen dosage form is dosed at any higher dose. These controlled release dosage forms thus maintain at least 80% COX-2 inhibition in canine blood for a period of time longer than that achieved by an immediate release dosage form at a higher dose.

Most preferred oral controlled release carprofen dosage forms are those which are able to maintain plasma carprofen levels above approximately 10 μg/mL for a period of time greater than or equal to the time observed for an immediate release 2 mg/lb carprofen dosage form (10.5 hrs), when said oral controlled release carprofen dosage forms are administered at a dose less than 2 mg/lb. The performance of a 2 mg/lb oral immediate release dosage form is taken as the fundamental standard for purposes of this comparison since 2 mg/lb/day is the currently recommended and accepted efficacious oral dose in accordance with the present invention as described herein.

As described below, controlled release oral dosage form carprofen release rates were calculated which result in canine carprofen plasma concentrations greater than 10 μg/mL. For ease of computation, these calculated rates were “zero order” rates; thus the computed rates were for controlled release devices which release carprofen at a constant (i.e. zero order) rate. It will be appreciated by those skilled in the art that practical dosage forms release at “zero order” for only a portion of their drug release time, whereafter they release at “first order”, or release at “mixed order”. In order to make the definition of carprofen delivery rates (or release rates) even more clear to the skilled artisan in the analysis below, useful release rates have been defined according to the time at which 80% of the carprofen has exited from the dosage form, regardless of the drug release mechanism involved.

Zero-order release rates resulting in simulated plasma carprofen concentrations above 10 μg/mL were determined by the method of Zhou and Notari. See Zhou, M. and N. Re, “Methodology for using oral dose pharmacokinetic data to select drugs for prolonged release formulations and validation of the method using simulated data”, Biopharm Drug Disp, 1995. 16, 319-331. Average data resulting from the oral administration of 2 mg/lb carprofen to canines in an immediate release formulation were fitted to the equation

C=Cᵢ(e^{-S}^{₂}^{t}−e^{-S}^{₂}^{t})

where Cᵢ, S2, and S. are parameters, and t is time. During zero-order release the plasma carprofen concentrations were simulated by the following equation:

C=(k₀Cᵢ/D_{ref})[(e^{-S}^{₁}^{t}−1)/S−(e^{-S}^{₂}^{t}−1)/S₂]

where k₀ is the zero-order release rate and D_{ref} is the reference dose. After release is completed, the following equation is applicable:

C=(k₀C/D_{ref})[[(1−e^{-S}^{₁}^{T})/S₁]e^{-S}^{₂}^{t}−[(1−e^{S}^{₂}^{T})/S₂]e^{-s}^{₂}^{t}]

where T=D_{CR}/k₀, and D_{CR} is the controlled release dose.

### Preferred Oral Controlled Release Carprofen Dosage Forms

Table 6 shown below demonstrates that oral controlled release carprofen dosage forms (2 mg/lb dose) which release 80% of their incorporated carprofen in a time period ranging from 1.6 to 19.2 hrs have the capacity to give carprofen plasma levels greater than 10 μg/mL for greater than 10.5 hrs. Accordingly, these are preferred dosage forms at 2 mg/lb.

Table 7 shown below demonstrates that oral controlled release carprofen dosage forms (1.8 mg/lb dose) which release 80% of their incorporated carprofen in a time period ranging from 1.6 to 19.2 hrs have the capacity to give carprofen plasma levels greater than 10 μg/mL for greater than 9.5 hrs. Accordingly, these are preferred dosage forms at 1.8 mg/lb.

Table 8 demonstrates that oral controlled release carprofen dosage forms (1.6 mg/lb dose) which release 80% of their incorporated carprofen in a time period ranging from 1.6 to 19.2 hrs have the capacity to give carprofen plasma levels greater than 10 μg/mL for greater tharL8.5 hrs. Accordingly, these are preferred dosage forms at 1.6 mg/lb.

Table 9 demonstrates that oral controlled release carprofen dosage forms (1.4 mg/lb dose) which release 80% of their incorporated carprofen in a time period ranging from 1.6 to 19.2 hrs have the capacity to give carprofen plasma levels greater than 10 Ag/mL for greater than 7.5 hrs. Accordinly, these are preferred dosage forms at 1.4 mg/lb.

### More Preferred Oral Carprofen Controlled Release Dosage Forms

Controlled release carprofen dosage forms of this invention are particularly useful because they are able to maintain carprofen plasma levels greater than 10 μg/mL for greater than 10.5 hrs, even when they are administered at doses lower than the preferred efficacious use dose of 2 mg/lb. Table 7 demonstrates that oral controlled release carprofen dosage forms (1.8 mg/lb dose) which release 80% of their incorporated carprofen in a time period ranging from 4.8 to 19.2 hrs have the capacity to give carprofen plasma levels greater than 10 gg/mL for greater than 10.5 hrs. These are more preferred dosage forms at a dose level of 1.8 mg/lb.

Table 8 demonstrates that oral controlled release carprofen dosage forms (1.6 mg/lb dose) which release 80% of their incorporated carprofen in a time period ranging from 4.8 to 19.2 hrs have the capacity to give carprofen plasma levels greater than 10 μg/mL for greater than 9.5 hrs, i.e., for longer than the duration which a 1.8 mg/lb immediate release dosage form is able to achieve. These are, consequently, more preferred dosage forms at a dose level of 1.6 mg/lb.

Table 9 demonstrates that oral controlled release carprofen dosage forms (1.4 mg/lb dose) which release 80% of their incorporated carprofen in a time period of 4.8 to 19.2 hrs have the capacity to give carprofen plasma levels greater than 10 ug/mL for greater than or equal to 8.5 hrs, i.e., for longer than the duration which a 1.6 mg/lb immediate release dosage form is able to achieve. These are, consequently, more preferred dosage forms at a dose level of 1.4 mg/lb.

### Most Preferred Oral Carprofen Controlled Release Dosage Forms

Most preferred oral controlled release carprofen dosage forms are those which are able to maintain plasma carprofen levels above approximately 10 pg/mL for a period of time greater than or equal to the time observed for an immediate release 2 mg/lb carprofen dosage form (10.5 hrs), when said oral controlled release carprofen dosage forms are administered at a dose of less than 2 mg/lb. The performance of a 2 mg/lb oral immediate release dosage form is taken as a fundamental standard for purposes of comparison since 2 mg/lb/day is the currently recommended efficacious oral dose in accordance with the present invention. The data in Tables 6-9 are used to define the characteristics of the most preferred oral controlled release dosage forms of the present invention.

At 1.6 mg/lb dose, most preferred oral controlled release carprofen dosage forms are those which release 80% of their incorporated carprofen over the range 6.4 to 19.2 hrs. At 1.4 mg/lb dose, most preferred oral controlled release carprofen dosage forms are those which release 80% of their incorporated carprofen over approximately 12.8 hrs (10-14 hrs).

TABLE 6
Number of hours for which carprofen plasma concentrations
are above 10 μg/mL, after dosing with controlled release
dosage forms which release carprofen at various rates
(computed); dose = 2 mg/lb.

| Zero-order | Time for 80% release | Duration (hrs) for which plasma |
|---|---|---|
| release rate | of carprofen from | carprofen concentration is |
| (mg/lb/hr) | dosage form (hrs) | above 10 μg/mL. |
| 0.084 | 19.2 | 17.50 |
| 0.100 | 16 | 16.5 |
| 0.125 | 12.8 | 15.5 |
| 0.165 | 9.6 | 13.5 |
| 0.250 | 6.4 | 12.5 |
| 0.330 | 4.8 | 12 |
| 1.0 | 1.6 | 11 |

TABLE 7
Number of hours for which carprofen plasma concentrations
are above 10 μg/mL, after dosing with controlled release
dosage forms which release carprofen at various rates
(computed); dose = 1.8 mg/lb.

| Zero-order | Time for 80% release | Duration (hrs) for which plasma |
|---|---|---|
| release rate | of carprofen from | carprofen concentration is |
| (mg/lb/hr) | dosage form (hrs) | above 10 μg/mL |
| 0.075 | 19.2 | 14 |
| 0.090 | 16 | 14.5 |
| 0.113 | 12.8 | 14.5 |
| 0.150 | 9.6 | 13 |
| 0.225 | 6.4 | 11.5 |
| 0.3 | 4.8 | 11 |
| 0.9 | 16 | 10 |

TABLE 8
Number of hours for which carprofen plasma concentrations
are above 10 μg/mL, after dosing with controlled release
dosage forms which release carprofen at various rates
(computed); dose = 1.6 mg/lb.

| Zero-order | Time for 80% release | Duration (hr) for which plasma |
|---|---|---|
| release rate | of carprofen from | carprofen concentration is |
| (mg/lb/hr) | dosage form (hr) | above 10 μg/mL |
| 0.067 | 19.2 | 11 |
| 0.080 | 16 | 13 |
| 0.100 | 12.8 | 12.5 |
| 0.135 | 9.6 | 11.5 |
| 0.2 | 6.4 | 11 |
| 0.27 | 4.8 | 10 |
| 0.8 | 1.6 | 9 |

TABLE 9
Number of hours for which carprofen plasma concentrations
are above 10 μg/mL, after dosing with controlled release
dosage forms which release carprofen at various rates
(computed). Dose = 1.4 mg/lb.

| Zero-order | Time for 80% release | Duration (hr) for which plasma |
|---|---|---|
| release rate | of carprofen from | carprofen concentration is |
| (mg/lb/hr) | dosage form (hr) | above 10 μg/mL |
| 0.059 | 19.2 | 0 |
| 0.07 | 16 | 9 |
| 0.088 | 12.8 | 10.5 |
| 0.115 | 9.6 | 10 |
| 0.175 | 6.4 | 9.5 |
| 0.23 | 4.8 | 8.5 |
| 0.7 | 1.6 | 8 |

### EXAMPLE 6

Carprofen Implants for Dogs.

Carprofen implants are useful for delivery of carprofen over extended time periods, e.g., 3 days, 7 days, 30 days, and so forth. This below-detailed example describes useful and preferred carprofen release rates from implants containing carprofen, and also defines doses associated therewith.

The input rate of carprofen into the canine body which would provide a steady state plasma carprofen concentration of 10 pig/mL was calculated using the following equation from Gibaldi, M. and D. Perrier, “Pharmacokinetics”, 2nd ed. Drugs and the Pharmaceutical Sciences, ed. J. Swarbrick, Vol. 15, 1982, New York: Marcel Dekker, Inc.:

$${C}_{ss} = \frac{{R}_{0}}{Cl}$$

where Cₛₛ is the steady state plasma carprofen concentration, R₀ is the input rate, and Cl is the systemic clearance. The systemic clearance was estimated from the pharmacokinetics observed after oral administration of 2 mg/lb carprofen to dogs, using the equation:

$$Cl = \frac{FD}{AUC}$$

where F is the bioavailability (assumed to be 1 for an implant), D is the oral dose, and AUC is the average area under the plasma carprofen concentration vs. time curve, extrapolated to infinity.

A clearance of Cl=5 mL/hr per lb body weight was obtained. Using this clearance, and a target Css of 10 μg/mL, a carprofen release rate Ro of 50 μg/lb/hr was calculated, which upon multiplication by 24 hr/day gives a daily input rate of 1.2 mg/lb/day. A total dose of 3.6 mg/lb or 8.4 mg/lb or 36 mg/lb would be required for 3, 7, or 30 day therapy, respectively. In summary, an implant containing 8.4 mg carprofen per lb body weight, releasing carprofen at a rate of 50 μg per lb of body weight per hr, maintains a plasma carprofen concentration of 10 μg/mL for 7 days. This will maintain 80% COX-2 inhibition for 7 days.

To obtain the implant dose and carprofen release rate required to maintain a plasma carprofen concentration of 2 ug/mL (50% COX-2 inhibition), the dose and release rate for a 10 gg/mL target are linearly adjusted by multiplication by 0.2. Thus for a target plasma carprofen concentration of 2 μg/mL, a carprofen release rate of 10 μg/lb/hr is needed, or 0.24 mg/lb/day. A total dose of 0.72 mg/lb or 1.68 mg/lb or 7.2 mg/lb would be required for 3, 7, or 30 day therapy, respectively.

To obtain the implant dose and carprofen release rate required to maintain a plasma carprofen concentration of 20 μg/mL (90% COX-2 inhibition), the dose and release rate for a 10 μg/mL target are linearly adjusted by multiplication by 2. Thus for a target plasma carprofen concentration of 20 μg/mL, a carprofen release rate of 100 μg/lb/hr is needed, or 2.4 mg/lb/day. A total dose of 7.2 mg/lb or 16.8 mg/lb or 72 mg/lb would be required for 3, 7, or 30 day therapy, respectively.

Useful carprofen implants of the present invention release carprofen into the canine body at a rate of 0.24 mg/lb/day or greater.

Preferred carprofen implants of this invention release carprofen into the canine body at a rate of 0.24 to 1.2 mg/lb/day.

More preferred carprofen implants of this invention release carprofen into the canine body at a rate of 1.2 to 2.4 mg/lb/day.

Useful carprofen implants have a total carprofen dose up to 2 gm, limited by the size of an implant which can be reasonably administered to a dog. Of course, more than one implant may be administered at the same time.

### EXAMPLE 7

Resolution of (S)-6-chloro-α-methyl-carbazole-2-acetic Acid

A solution of 4.3 g of (R)-α-methylbenzylamine in 20 mL of acetone was added to a solution of 9.7 g of partially resolved 6-chloro-α-methyl-carbazole-2-acetic acid (recovered from filtration of a previous resolution of the racemate). After standing at room temperature for 24 hrs, the mixture was filtered and the filter cake was washed with cold acetone to yield after drying 7.3 g. Following two additional recrystallizations from acetone, 1.9 g of (S)-6-chloro-α-methyl-carbazole-2-acetic acid (R)-α-methylbenzylamine salt, [α]_{η}²²−13.60 was obtained. Further recrystallizations from acetone did not change the rotation. The salt was dissolved in 50 mL of warm acetone and the solution after filtration was poured into 500 mL of dilute hydrochloric acid. Following filtration and drying, 1.4 g was obtained, which upon crystallization from chloroform gave 0.9 g. of (S)-6-chloro-α-methyl-carbazole-2-acetic acid, m.p. 198°-201°, [α]_{η}^{22+53.2°}, (c 1.33, CH₃OH).

### EXAMPLE 8

Species Specificity of COX-2 Selectivity: Activity in Members of the Species Canis familiaris (Dogs) Compared to Activity in Members of the Species Rattus norvegicus (White Rats) and in Homo sapiens (Humans)

The very high degree of COX-2 selectivity exhibited by carprofen in dogs has already been amply demonstrated in Example 1. Equally surprising was the discovery that this selective inhibition of the COX-2 enzyme appears to be an activity which is specific to the species Canis familiaris, and not shared by other species. This discovery was based on the evaluation of the inhibitory activity of racemic carprofen in members of the species Rattus norvegicus (white rats) and in members of the species Homo sapiens (humans).

In vivo cyclo-oxygenase selectivity was evaluated in rats by the method of Griffiths et al., described in Agents & Actions, 32, (1991), 313-320. The COX-2 inhibitory activity was evaluated in accordance with the effect of racemic carprofen on prostaglandin PGE₂ production as measured in the synovial fluid of the rat. Synovial fluid is secreted by the synovial membrane and is contained in joint cavities. During joint inflammation, COX-2 is induced in joint tissues and prostaglandin products accumulate in the synovial fluid. The COX-1 inhibitory activity was evaluated in accordance with the effect of racemic carprofen on prostaglandin PGE₂ production as measured in the mucosal lining of the rat stomach, which contains significant amounts of the constitutive COX-1 isozyme. Inhibition of this stomach isozyme results in adverse gastrointestinal side effects. The COX-1 ED₅₀ was 6.4 mg/kg, while the COX-2 ED₅₀ was 0.63 mg/kg. These results indicate that in rats, there is only 10-fold selectivity for the COX-2 isozyme by racemic carprofen.

For humans, the COX-2 inhibitory activity was evaluated in accordance with the effect of racemic carprofen on levels of COX-2 in human umbilical vein endothelial cells (HUVEC) stimulated by IL-1 and phorbol myristate acetate (PMA) in accordance with the method of Habib et al. described in J. Biol. Chem., 268, 23448-23454, 1993. These endothelial cells under the stimulation of interleukin-1 (IL-1) and PMA are most likely to contain significant amounts of the inducible COX-2 isozyme. The COX-1 inhibitory activity was evaluated in accordance with the effect of racemic carprofen on levels of COX-1 as measured by a human washed platelets (HWP) TXB₂ biochemical assay, in accordance with the procedures of Grossman, et al. described in Inflamm. Res., 44, 253-257, 1995. These platelets are most likely to contain significant amounts of the constitutive COX-1 isozyme. The HUVEC (COX-2) IC₅₀ (μM) was 1.20, while the HWP TXB₂ (COX-1) IC₅₀ (μM) was 0.77. These results indicate that in humans, there is no selectivity for the COX-2 isozyme by racemic carprofen.

### EXAMPLE 9

Tablet Formulation of (S)-6-chloro-α-methyl-carbazole-2-acetic Acid

Tablet Formulation

| Ingredients | Weight per Tablet |
|---|---|
| (S)-6-chloro-α-methyl-carbazole-2-acetic | 25.00 mg |
| acid | |
| Lactose, U.S.P. | 64.50 mg |
| Corn Starch | 10.00 mg |
| Magnesium Stearate | 0.50 mg |

### EXAMPLE 10

Capsule Formulation of (S)-6-chloro-α-methyl-carbazole-2-acetic Acid

Capsule Formulation

| Ingredients | Weight per Capsule |
|---|---|
| (S)-6-chloro-α-methyl-carbazole-2-acetic | 50 mg |
| acid | |
| Lactose, U.S.P. | 124 mg |
| Corn Starch, U.S.P. | 30 mg |
| Talc, U.S.P. | 5 mg |
| Total Weight | 210 mg |

### EXAMPLE 11

Parenteral Formulation of (S)-6-chloro-α-methyl-carbazole-2-acetic Acid

Parenteral Formulation

| Ingredients per 1 cc ampule | Weight per Ampule |
|---|---|
| (S)-6-chloro-α-methyl-carbazole-2-acetic | 10.2 mg |
| acid | |
| Methyl Paraben, U.S.P. | 1.8 mg |
| Propyl Paraben, U.S.P. | 0.2 mg |
| Sodium Hydroxide, U.S.P. q.s. ph | 9.0 mg |
| Water for Injection, U.S.P. q.s. ad | 1.0 cc |

What is claimed is:

1. A method of treating or preventing pain and inflammatory processes and diseases associated with the activity of inducible cyclo-oxygenase-2 (COX-2) in a member of the species Canis familiaris in need of such treatment, while at the same time reducing or eliminating undesirable side effects associated with simultaneous inhibition of the activity of constitutive cyclo-oxygenase-1 (COX-1) by selectively inhibiting COX-2 activity with reference to COX-1 activity, wherein the selectivity ratio of COX-2:COX-1 activity inhibition is at least 3:1 based on ex vivo inhibition levels in whole blood measured at a dose giving ≧80% COX-2 inhibition, comprising administering to said member of the species Canis familiaris an amount therapeutically effective for treating pain and inflammation in accordance with the above-recited limitations, of an anti-inflammatory selective COX-2 inhibitory compound comprising a member selected independently from the group of anti-inflammatory compounds consisting essentially of salicylic acid derivatives; P-aminophenol derivatives; indole and indene acetic acids; heteroaryl acetic acids; arylpropionic acids; anthranilic acids; enolic acids; and alkanones.

2. A method of treating or preventing pain and inflammatory processes and diseases according to claim 1 wherein said anti-inflammatory selective COX-2 inhibitory compound comprises a compound of the formula: 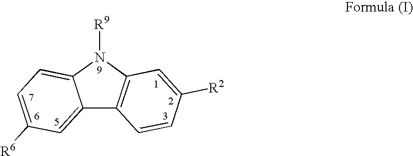
wherein: 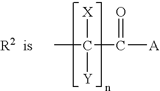
where A is hydroxy, (C₁-C₄)alkoxy, amino, hydroxyamino, mono-(C₁-C₂)alkylamino, di-(C₁-C₂)alkylamino; X and Y are independently H or (C₁-C₂)alkyl; and n is 1 or 2;
R⁶ is halogen, (C₁-C₃)alkyl, trifluoromethyl, or nitro;
R⁹ is H; (C₁-C₂)alkyl; phenyl or phenyl-(C₁-C₂)alkyl, where phenyl is optionally mono-substituted by fluoro or chloro; —C(═O)—R, where R is (C₁-C₂)alkyl or phenyl, optionally mono-substituted by fluoro or chloro; or —C(═O)—O—R¹, where R¹ is (C₁-C₂)alkyl;
where X and Y′ are different, the (−)(R) and (+)(S) enantiomers thereof; and all pharmaceutically acceptable salt forms, prodrugs and metabolites thereof which are therapeutically active for treating or preventing pain and inflammation.

3. A method of treating or preventing pain and inflammatory processes and diseases according to claim 2 wherein said anti-inflammatory selective COX-2 inhibitory compound is 6-chloro-α-methyl-9H-carbazole-2-acetic acid.

4. A method of treating or preventing pain and inflammatory processes and diseases according to claim 2 wherein said anti-inflammatory selective COX-2 inhibitory compound is comprised entirely of (S)-enantiomer of 6-chloro-(X-methyl-9H-carbazole-2-acetic acid.

5. A method of treating or preventing inflammatory processes and diseases as in claims 1, 2, 3, or 4 further comprising wherein said inhibitory compound is used in combination with one or more other therapeutically active agents under the following conditions:
A. where a joint has become seriously inflammed as well as infected at the same time by bacteria, fungi, protozoa, and/or virus, said inhibitory compound is administered in combination with one or more antibiotic, antifungal, antiprotozoal, and/or antiviral therapeutic agents;
B. where a multi-fold treatment of pain and inflammation is desired, said inhibitory compound is administered in combination with inhibitors of other mediators of inflammation, comprising one or more members independently selected from the group consisting essentially of:
1. NSAIDs;
2. H₁-receptor antagonists;
3. kinin-B₁- and B₂-receptor antagonists;
4. prostaglandin inhibitors selected from the group consisting of PGD-, PGF-PGI₂-, and PGE-receptor antagonists;
5. thromboxane A₂ (TXA2-) inhibitors;
6. 5- and 12-lipoxygenase inhibitors;
7. leukotriene LTC₄-, LTD₄/LTE₄-, and LTB₄-inhibitors;
8. PAF-receptor antagonists;
9. gold in the form of an aurothio group together with one or more hydrophilic groups;
10. immunosuppressive agents selected from the group consisting of cyclosporine, azathioprine, and methotrexate;
11. anti-inflammatory glucocorticoids;
12. penicillamine;
13. hydroxychloroquine;
14. anti-gout agents including colchicine; xanthine oxidase inhibitors including allopurinol; and uricosuric agents selected from probenecid, sulfinpyrazone, and benzbromarone;
C. where older dogs are being treated for disease conditions, syndromes and symptoms found in geriatric dogs, said inhibitory compound is administered in combination with one or more members independently selected from the group consisting essentially of:
1. cognitive therapeutics to counteract memory loss and impairment;
2. anti-hypertensives and other cardiovascular drugs intended to offset the consequences of atherosclerosis, hypertension, myocardial ischemia, angina, congestive heart failure, and myocardial infarction, selected from the group consisting of:
a. diuretics;
b. vasodilators;
c. β-adrenergic receptor antagonists;
d. angiotensin-II converting enzyme inhibitors (ACE-inhibitors), alone or optionally together with neutral endopeptidase inhibitors;
e. angiotensin 11 receptor antagonists;
f. renin inhibitors;
g. calcium channel blockers;
h. sympatholytic agents;
i. α₂-adrenergic agonists;
j. a-adrenergic receptor antagonists; and
k. HMG-CoA-reductase inhibitors (anti-hypercholester-olemics);
3. antineoplastic agents selected from:
a. antimitotic drugs selected from:
i. vinca alkaloids selected from:
[1] vinblastine, and
[2] vincristine;
4. growth hormone secretagogues;
5. strong analgesics;
6. local and systemic anesthetics; and
7. H₂-receptor antagonists, proton pump inhibitors, and other gastroprotective agents.

6. A pharmaceutical composition for treating or preventing pain and inflammatory processes and diseases associated with the activity of inducible cyclo-oxygenase-2 (COX-2) in a member of the species Canis familiaris in need of such treatment, while at the same time reducing or eliminating undesirable side effects associated with simultaneous inhibition of the activity of constitutive cyclo-oxygenase-1 (COX-1), comprising:
A. a therapeutically effective amount for treating pain and inflammation, of an anti-inflammatory selective COX-2 inhibitory compound which selectively inhibits COX-2 activity with reference to COX-1 activity, wherein the selectivity ratio of COX-2: COX-1 activity inhibition is at least 3:1 based on ex vivo inhibition levels in whole blood measured at a dose giving ≧80% COX-2 inhibition, comprising a member independently selected from the group of anti-inflammatory compounds consisting essentially of salicylic acid derivatives; p-aminophenol derivatives; indole and indene acetic acids; heteroaryl acetic acids; arylpropionic acids; anthranilic acids; enolic acids; and alkanones; and
B. a pharmaceutically acceptable carrier therefor.

7. A pharmaceutical composition according to claim 6 wherein said anti-inflammatory selective COX-2 inhibitory compound comprises a compound of the formula: 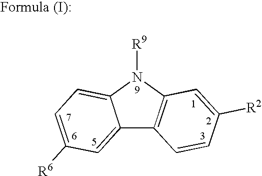
wherein: 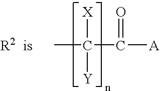
where A is hydroxy, (C₁C₄)alkoxy, amino, hydroxyamino, mono-(C₁-C₂)alkylamino, di-(C₁-C₂)alkylamino; X and Y are independently H or (C₁-C₂)alkyl; and n is 1 or 2;
R⁶ is halogen, (C₁-C₃)alkyl, trifluoromethyl, or nitro;
R⁹ is H; (C₁-C₂)alkyl; phenyl or phenyl-(C₁-C₂)alkyl, where phenyl is optionally mono-substituted by fluoro or chloro; —C(═O)—R, where R is (C₁-C₂)alkyl or phenyl, optionally mono-substituted by fluoro or chloro; or —C(═O)—O—R¹, where R¹ is (C₁-C₂)alkyl;
where X and Y are different, the (−)(R) and (+)(S) enantiomers thereof; and all pharmaceutically acceptable salt forms, prodrugs and metabolites thereof which are therapeutically active for treating or preventing pain and inflammation.

8. A pharmaceutical composition according to claim 7 wherein said anti-inflammatory selective COX-2 inhibitory compound is 6-chloro-Q-methyl-9H-carbazole-2-acetic acid.

9. A pharmaceutical composition according to claim 7 wherein said anti-inflammatory selective COX-2 inhibitory compound is comprised entirely of (S)-enantiomer of 6-chloro-α-methyl-9H-carbazole-2-acetic acid.

10. A pharmaceutical composition as in claims 6, 7, 8 or 9 further comprising wherein said anti-inflammatory selective COX-2 inhibitory compound is provided in a dosage form suitable for systemic administration by:
A. injection or infusion in suitable liquid form which is intraarterial, intra- or transdermal, subcutaneous, intramuscular, intraspinal, intrathecal, or intravenous, wherein said inhibitory compound:
1. is contained in solution as a solute;
2. is contained in the discontinuous phase of an emulsion, or the discontinuous phase of an inverse emulsion which inverts upon injection or infusion, said emulsions containing suitable emulsifying agents; or
3. is contained in a suspension as a suspended solid in colloidal or microparticulate form, said suspension containing suitable suspending agents;
B. injection or infusion into suitable body tissues or cavities as a depot, wherein said composition provides storage of said inhibitor and thereafter delayed-, sustained-, and/or controlled-release of said inhibitory compound for systemic distribution;
C. instillation, inhalation or insufflation into suitable body tissues or cavities in suitable solid form, where said inhibitory compound:
1. is contained in a solid implant composition providing delayed-, sustained- and/or controlled-release of said inhibitory compound;
2. is contained in a particulate composition to be inhaled into the lungs; or
3. is contained in a particulate composition to be blown into said suitable body tissues or cavities, wherein said composition optionally provides delayed-, sustained-, and/or controlled-release of said inhibitory compound; or
D. ingestion in suitable solid or liquid form for peroral delivery of said inhibitory compound, where said inhibitory compound:
1. is contained in a solid dosage form; or
2. is contained in a liquid dosage form.

11. A pharmaceutical composition according to claim 10 wherein said dosage forms comprise one or more members selected independently from the group consisting essentially of suppositories; solid peroral dosage forms selected from the group consisting of delayed-release tablets, capsules, caplets, lozenges, troches, and multiparticulates; enteric-coated tablets and capsules which prevent release and absorption in the stomach of said member being treated to facilitate delivery distal to the stomach of said member; sustained-release oral tablets, capsules and microparticulates which provide systemic delivery of said inhibitor in a controlled manner over at least a 10-hour period; a chewable or ingestible oral tablet; a unit dose packet sachet, a suspension made from said unit dose packet sachet, a powder for oral suspension, or an oral suspension per se; a fast-dissolving tablet; encapsulated solutions; an oral paste; a granular form incorporated in or to be incorporated in said member's food; and a palatable chewable form in which said inhibitor is consumed along with said palatable chewable form, or is delivered by leaching from said chew, which is not consumed, during mastication by said member being treated; liquid peroral dosage forms selected from the group consisting of solutions, suspensions, emulsions, inverse emulsions, elixirs, extracts, tinctures, and concentrates; and the above-recited solid dosage forms containing microencapsulated formulations of the active ingredient, which is incorporated into said solid dosage form.

12. A pharmaceutical composition as in claim 11 comprising an oral controlled release carprbfen dosage form able to maintain plasma carprofen levels above approximately 10 μg/mL for a period of time greater than 10.5 hours, when administered at a dose of about 2 mg/lb or less.

13. A pharmaceutical composition as in claims 6, 7, 8, or 9 further comprising said anti-inflammatory selective COX-2 inhibitory compound in combination with one or more other therapeutically active agents independently selected from the group consisting essentially of:
A. anti-infectious agents comprising one or more antibiotic, antifungal, antiprotozoal, or antiviral therapeutic agents,
B. inhibitors of other mediators of inflammation, comprising one or more members independently selected from the group consisting essentially of:
1. NSAIDs;
2. H₁-receptor antagonists;
3. kinin-B₁- and B₂-receptor antagonists;
4. prostaglandin inhibitors selected from the group consisting of PGD-, PGF-PGl₂-, and PGE-receptor antagonists;
5. thromboxane A₂ (TXA2-) inhibitors;
6. 5- and 12-lipoxygenase inhibitors;
7. leukotriene LTC₄-LTD₄/LTE₄-, and LTB₄-inhibitors;
8. PAF-receptor antagonists;
9. gold in the form of an aurothio group together with one or more hydrophilic groups;
10. immunosuppressive agents selected from the group consisting of cyclosporine, azathioprine, and methotrexate;
11. anti-inflammatory glucocorticoids;
12. penicillamine;
13. hydroxychloroquine;
14. anti-gout agents including colchicine; xanthine oxidase inhibitors including allopurinol; and uricosuric agents selected from probenecid, sulfinpyrazone, and benzbromarone;
C. therapeutic agents for the treatment of geriatric dogs comprising one or more members independently selected from the group consisting essentially of:
1. cognitive therapeutics to counteract memory loss and impairment;
2. anti-hypertensives and other cardiovascular drugs intended to offset the consequences of atherosclerosis, hypertension, myocardial ischemia, angina, congestive heart failure, and myocardial infarction, selected from the group consisting of:
a. diuretics;
b. vasodilators;
c. P-adrenergic receptor antagonists;
d. angiotensin-II converting enzyme inhibitors (ACE-inhibitors), alone or optionally together with neutral endopeptidase inhibitors;
e. angiotensin 11 receptor antagonists;
f. renin inhibitors;
g. calcium channel blockers;
h. sympatholytic agents;
i. α₂-adrenergic agonists;
j. α-adrenergic receptor antagonists; and
k. HMG-CoA-reductase inhibitors (anti-hypercholester-olemics);
3. antineoplastic agents selected from:
a. antimitotic drugs selected from:
i. vinca alkaloids selected from:
[1] vinblastine, and
[2] vincristine;
4. growth hormone secretagogues;
5. strong analgesics;
6. local and systemic anesthetics; and
7. H₂-receptor antagonists, proton pump inhibitors, and other gastroprotective agents.

14. A package suitable for use in commerce for the therapeutic treatment or prevention of pain and inflammation processes and diseases in a member of the species Canis familiaris in need of such treatment, comprising:
A. a suitable container optionally in the form of an outer package and an inner container removably housed therein;
B. a suitable dosage form, enclosed in said container, of an anti-inflammatory selective COX-2 inhibitory compound of the formula: 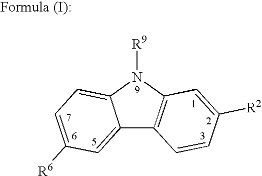
wherein: 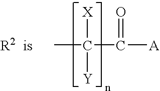
where A is hydroxy, (C₁-C₄)alkoxy, amino, hydroxyamino, mono-(C₁-C₂)alkylamino, di-(C₁-C₂)alkylamino; one of X and Y is H and the other is (C₁-C₂)alkyl; and n is 1 or 2;
R⁶ is halogen, (C₁-C₃)alkyl, trifluoromethyl, or nitro;
R⁹ is,H; (C₁-C₂)alkyl; phenyl or phenyl-(C₁-C₂)alkyl, where phenyl is optionally mono-substituted by fluoro or chloro; —C(═O)—R, where R is (C₁-C₂)alkyl or phenyl, optionally mono-substituted by fluoro or chloro; or —C(═O)—O—R¹, where R¹ is (C₁-C₂)alkyl;
wherein (+)(S) enantiomer is present in amount of at least 75%; and all pharmaceutically acceptable salt forms, prodrugs and metabolites thereof which are therapeutically active for treating or preventing pain and inflammation; and
C. printed instructional and informational material associated with said container, which is attached to said container, enclosed in said container, or displayed as an integral part of said container, said instructional and informational material stating in words which convey to a reader thereof of ordinary skill in the art that said compound of Formula (I) comprising a therapeutic agent contained in said package, when administered to said member of the species Canis familiaris to be treated, effectively inhibits cyclo-oxygenase-2 (COX-2) induced at an existing or expected site of pain and inflammation in said dog, thereby treating or preventing said pain and inflammation which would otherwise result therefrom, while at the same time reducing or eliminating undesirable side effects associated with simultaneous inhibition of the activity of constitutive cyclo-oxygenase-1 (COX-1) by selectively inhibiting COX-2 activity with reference to COX-1 activity, wherein the selectivity ratio of COX-2: COX-1 activity inhibition is at least 3:1 based on ex vivo inhibition levels in whole blood measured at a dose giving >80% COX-2 inhibition.

15. A package according to claim 14 wherein said anti-inflammatory selective COX-2 inhibitory compound of Formula (I) comprises 6-chloro-α-methyl-9H-carbazole-2-acetic acid.

16. A package according to claim 14 wherein said anti-inflammatory selective COX-2 inhibitory compound is comprised entirely of (S)-enantiomer of 6-chloro-α-methyl-9H-carbazole-2-acetic acid.
